(19) United States
(12) Patent Application Publication
(10) Pub. No.: US 2015/0335609 A1
(43) Pub. Date: Nov. 26, 2015

(54) COMBINATIONS OF HISTONE DEACETYLASE INHIBITOR AND PAZOPANIB AND USES THEREOF

(71) Applicant: Pharmacyclics, Inc., Sunnyvale, CA (US)

(72) Inventors: Sriram BALASUBRAMANIAN, San Carlos, CA (US); Tarak D. MODY, Sunnyvale, CA (US)

(21) Appl. No.: 14/377,380

(22) PCT Filed: Feb. 15, 2013
(86) PCT No.: PCT/US2013/026462

§ 371 Date: Aug. 7, 2014

Related U.S. Application Data
(60) Provisional application No. 61/600,491, filed on Feb. 17, 2012.
(60) Provisional application No. 61/602,544, filed on Feb. 23, 2012.

(51) Int. Cl. A61K 31/343; A61K 45/06; A61N 5/10; A61K 31/506

(52) U.S. Cl. CPC A61K 31/343; A61K 31/506; A61K 45/06; A61N 5/10

(57) ABSTRACT

Dosing regimens, methods of treatment, controlled release formulations, and combination therapies that include an HDAC inhibitor, or a pharmaceutically acceptable salt thereof, and pazopanib (or a salt thereof, e.g., pazopanib HCI) are described.

### CROSS-REFERENCE

This application claims the benefit of U.S. Provisional Patent Application Ser. No. 61/600,491, filed Feb. 17, 2012, and U.S. Provisional Patent Application Ser. No. 61/602,544, filed Feb. 23, 2012, both of which are incorporated herein in their entirety by reference.

### BACKGROUND OF THE INVENTION

The acetylation state of nucleosomal histones regulates gene expression. Deacetylation of nucleosomal histones is catalyzed by a group of enzymes known as histone deacetylases (HDACs), of which there are eleven known isoforms. Histone deacetylation leads to chromatin condensation resulting on transcriptional repression, whereas acetylation induces localized relaxation within specific chromosomal regions to allow better access to transcriptional machinery to facilitate transcription.

In tumor cells, use of selective inhibitors of HDAC enzymes has been reported to result in histone hyperacetylation. This alters the transcriptional regulation of a subset of genes, including many tumor suppressors, genes involved in cell cycle control, cell division and apoptosis. Further, HDAC inhibitors have been reported to inhibit tumor growth in vivo. The inhibition of tumor growth is accompanied by histone and tubulin hyperacetylation and may involve multiple mechanisms.

HDAC inhibitors block cancer cell proliferation both in vitro and in vivo. N-hydroxy-4-{2-[3-(N,N-dimethylaminomethyl)benzofuran-2-ylcarbonylamino]ethoxy}-benzamide (also known as PCI-24781 or abexinostat) is a hydroxamate-based HDAC inhibitor for use in the treatment of cancer in a human.

### SUMMARY OF THE INVENTION

Disclosed herein, in certain embodiments, are methods of increasing the effectiveness of an antiangiogenic agent in an individual in need thereof, comprising co-administering to the individual (a) a cycle of abexinostat or a salt thereof, and (b) an antiangiogenic agent. In some embodiments, the antiangiogenic agent is pazopanib or a salt thereof. In some embodiments, the method reduces resistance to the antiangiogenic agent; delays the development of resistance to the antiangiogenic agent; delays the onset of the cancer becoming refractory to the antiangiogenic agent; prolongs the usefulness of the antiangiogenic agent; allows use of the antiangiogenic agent in the treatment of cancers that generally develop, or have developed, resistance to the antiangiogenic agent; increases patient response to the antiangiogenic agent; increases cellular response to the antiangiogenic agent; decreases the effective dosage of the antiangiogenic agent; or any combination thereof. In some embodiments, the salt of abexinostat is abexinostat HCl. In some embodiments, abexinostat, or a salt thereof, and the antiangiogenic agent are administered separately, concurrently or sequentially. In some embodiments, the subject is in an interdigestive state. In some embodiments, the abexinostat, or a salt thereof, and the antiangiogenic agent, are administered one hour before a meal or 2 hours after a meal. In some embodiments, the cycle of abexinostat, or a salt thereof, is 5 days. In some embodiments, at least one dose of abexinostat, or a salt thereof, is administered each day of the abexinostat cycle. In some embodiments, the dose of abexinostat, or a salt thereof, is sufficient to maintain an effective plasma concentration of abexinostat, or the salt thereof, in the individual for at least about 6 consecutive hours to about 8 consecutive hours. The method of claim 2, comprising administering a first dose of abexinostat, or a salt thereof, and a second dose of abexinostat, or a salt thereof, 4 to 8 hours apart. In some embodiments, the cancer is a hematological cancer, solid tumor or a sarcoma. In some embodiments, the cancer is a solid tumor. In some embodiments, the cancer is a metastatic solid tumor or an advanced solid tumor. In some embodiments, the cancer is a sarcoma. In some embodiments, the cancer is soft tissue sarcoma. In some embodiments, the cancer is renal cell carcinoma or ovarian cancer. In some embodiments, the method further comprises administering at least one additional therapy selected from anti-cancer agents, anti-emetic agents, radiation therapy, or combinations thereof.

Disclosed herein, in certain embodiments, are methods of treating a cancer in an individual in need thereof, comprising co-administering to the individual (a) a cycle of abexinostat or a salt thereof, and (b) an antiangiogenic agent. In some embodiments, the antiangiogenic agent is pazopanib or a salt thereof. In some embodiments, the method reduces resistance to the antiangiogenic agent; delays the development of resistance to the antiangiogenic agent; delays the onset of the cancer becoming refractory to the antiangiogenic agent; prolongs the usefulness of the antiangiogenic agent; allows use of the antiangiogenic agent in the treatment of cancers that generally develop, or have developed, resistance to the antiangiogenic agent; increases patient response to the antiangiogenic agent; increases cellular response to the antiangiogenic agent; decreases the effective dosage of the antiangiogenic agent; or any combination thereof. In some embodiments, the salt of abexinostat is abexinostat HCl. In some embodiments, abexinostat, or a salt thereof, and the antiangiogenic agent are administered separately, concurrently or sequentially. In some embodiments, the subject is in an interdigestive state. In some embodiments, the abexinostat, or a salt thereof, and the antiangiogenic agent, are administered one hour before a meal or 2 hours after a meal. In some embodiments, the cycle of abexinostat, or a salt thereof, is 5 days. In some embodiments, at least one dose of abexinostat, or a salt thereof, is administered each day of the abexinostat cycle. In some embodiments, the dose of abexinostat, or a salt thereof, is sufficient to maintain an effective plasma concentration of abexinostat, or the salt thereof, in the individual for at least about 6 consecutive hours to about 8 consecutive hours. In some embodiments, the method further comprises a first dose of abexinostat, or a salt thereof, and a second dose of abexinostat, or a salt thereof, 4 to 8 hours apart. In some embodiments, the cancer is a hematological cancer, solid tumor or a sarcoma. In some embodiments, the cancer is a solid tumor. In some embodiments, the cancer is a metastatic solid tumor or an advanced solid tumor. In some embodiments, the cancer is a sarcoma. In some embodiments, the cancer is soft tissue sarcoma. In some embodiments, the cancer is renal cell carcinoma or ovarian cancer. In some embodiments, the cancer is resistant to the antiangiogenic agent; partially resistant to the antiangiogenic agent; or refractory to the antiangiogenic agent. In some embodiments, the method further comprises administering at least one additional therapy selected from anti-cancer agents, anti-emetic agents, radiation therapy, or combinations thereof.

Disclosed herein, in certain embodiments, are methods of treating a cancer in an individual in need thereof, comprising: administering (a) a cycle of abexinostat (or a salt thereof), and (b) pazopanib (or a salt thereof). In some embodiments, abexinostat (or a salt thereof) and pazopanib (or a salt thereof) are administered separately. In some embodiments, abexinostat (or a salt thereof) and pazopanib (or a salt thereof) are administered concurrently or sequentially. In some embodiments, the cycle of abexinostat (or a salt thereof) is 1 to 14 consecutive days, 2 to 14 consecutive days, 3 to 14 consecutive days, 4 to 14 consecutive days, 5 to 14 consecutive days, 6 to 14 consecutive days, 7 to 14 consecutive days, 8 to 14 consecutive days, 9 to 14 consecutive days, 10 to 14 consecutive days, 11 to 14 consecutive days, 12 to 14 consecutive days, or 13 to 14 consecutive days. In some embodiments, the cycle of abexinostat (or a salt thereof) is 2 consecutive days, 3 consecutive days, 4 consecutive days, 5 consecutive days, 6 consecutive days, 7 consecutive days, 8 consecutive days, 9 consecutive days, 10 consecutive days, 11 consecutive days, 12 consecutive days, 13 consecutive days, or 14 consecutive days. In some embodiments, the methods further comprise an abexinostat (or a salt thereof) drug holiday following an abexinostat (or a salt thereof) cycle. In some embodiments, the abexinostat (or a salt thereof) drug holiday is 1 to 14 consecutive days, 2 to 14 consecutive days, 3 to 14 consecutive days, 4 to 14 consecutive days, 5 to 14 consecutive days, 6 to 14 consecutive days, 7 to 14 consecutive days, 8 to 14 consecutive days, 9 to 14 consecutive days, 10 to 14 consecutive days, 11 to 14 consecutive days, 12 to 14 consecutive days, or 13 to 14 consecutive days. In some embodiments, the abexinostat (or a salt thereof) drug holiday is 2 consecutive days, 3 consecutive days, 4 consecutive days, 5 consecutive days, 6 consecutive days, 7 consecutive days, 8 consecutive days, 9 consecutive days, 10 consecutive days, 11 consecutive days, 12 consecutive days, 13 consecutive days, or 14 consecutive days. In some embodiments, at least one dose of abexinostat (or a salt thereof) is administered each day of the abexinostat cycle. In some embodiments, the dose of abexinostate is sufficient to maintain an effective plasma concentration of abexinostat (or a salt thereof) in the individual for at least about 6 consecutive hours. In some embodiments, the dose of abexinostat (or a salt thereof) is sufficient to maintain an effective plasma concentration of abexinostat (or a salt thereof) in the individual for at least about 8 consecutive hours. In some embodiments, the dose of abexinostat (or a salt thereof) is sufficient to maintain an effective plasma concentrations of abexinostat (or a salt thereof) in the individual for about 6 consecutive hours to about 8 consecutive hours. In some embodiments, the methods comprise administering a first dose of abexinostat (or a salt thereof) and a second dose of abexinostat (or a salt thereof), wherein the first dose and the second dose are administered 4 to 8 hours apart. In some embodiments, the methods comprise administering a first dose of abexinostat (or a salt thereof), a second dose of abexinostat (or a salt thereof) and a third dose of abexinostat (or a salt thereof), wherein the first dose, the second dose and the third dose are administered 4 to 8 hours apart. In some embodiments, abexinostate (or a salt thereof) is formulated as an oral dosage form. In some embodiments, abexinostate (or a salt thereof) is formulated as an immediate release oral dosage form or a controlled release oral dosage form. In some embodiments, the methods comprise administering a first immediate release oral dosage form comprising abexinostat (or a salt thereof) and a second immediate release oral dosage form comprising abexinostat (or a salt thereof), wherein the second immediate release oral dosage form is administered about 4 to about 8 hours form the first immediate release oral dosage form. In some embodiments, the oral dosage form completely releases abexinostat (or a salt thereof) over a period of about 2 hours to about 10 hours after administration. In some embodiments, the methods comprise administering abexinostat (or a salt thereof) in fast mode. In some embodiments, the methods comprise administering pazopanib (or a salt thereof) in fast mode. In some embodiments, the methods comprise administering abexinostat (or a salt thereof) one hour before a meal or 2 hours after a meal. In some embodiments, the methods comprise administering pazopanib (or a salt thereof) one hour before a meal or 2 hours after a meal. In some embodiments, the methods comprise administering between about 30 mg/m² and about 75 mg/m² of abexinostat (or a salt thereof) BID. In some embodiments, a daily dose of abexinostat (or a salt thereof) is between about 60 mg/m² and about 150 mg/m². In some embodiments, the methods comprise administering between about 400 mg and about 800 mg of pazopanib. In some embodiments, the salt of abexinostat is abexinostat HCl. In some embodiments, the salt of pazopanib is pazopanib HCl. In some embodiments, the methods comprise administering between about 433.4 mg and about 866.8 mg of pazopanib HCl. In some embodiments, the cancer is a hematological cancer, solid tumor or a sarcoma. In some embodiments, the cancer is a sarcoma. In some embodiments, the cancer is soft tissue sarcoma. In some embodiments, the cancer is selected from a: breast cancer, colon cancer, colorectal cancer, non-small cell lung cancer, small-cell lung cancer, liver cancer, ovarian cancer, prostate cancer, uterine cervix cancer, urinary bladder cancer, gastric cancer, gastrointestinal stromal tumor, pancreatic cancer, germ cell tumor, mast cell tumor, neuroblastoma, mastocytosis, testicular cancer, glioblastoma, astrocytoma, B cell lymphoma, T cell lymphoma, Hodgkin's lymphoma, non-Hodgkin's lymphoma, melanoma, myeloma, acute myelocytic leukemia (AML), acute lymphocytic leukemia (ALL), myelodysplastic syndrome, chronic myelogenous leukemia, and renal cell carcinoma. In some embodiments, the cancer is selected from: breast cancer, colon cancer, colorectal carcinomas, non-small cell lung cancer, liver cancer, ovarian cancer, uterine cervix cancer, gastric carcinoma, pancreatic cancer, glioblastomas, B cell lymphoma, T cell lymphoma, Hodgkin's lymphoma, non-Hodgkin's lymphoma, myeloma, myelodysplastic syndrome (MDS), and renal cell carcinoma. In some embodiments, the cancer is renal cell carcinoma or ovarian cancer. In some embodiments, the method further comprises administering at least one additional therapy selected from anti-cancer agents, anti-emetic agents, radiation therapy, or combinations thereof. In some embodiments, the method further comprises administering at least one additional therapeutic agent selected from among DNA-damaging agents; topoisomerase I or II inhibitors; alkylating agents; PARP inhibitors; proteasome inhibitors; RNA/DNA antimetabolites; antimitotics; immunomodulatory agents; antiangiogenics; aromatase inhibitors; hormone-modulating agents; apoptosis inducing agents; kinase inhibitors; monoclonal antibodies; abarelix; ABT-888; aldesleukin; aldesleukin; alemtuzumab; alitretinoin; allopurinol; altretamine; amifostine anastrozole; arsenic trioxide; asparaginase; azacitidine; AZD-2281; bendamustine; bevacizumab; bexarotene; bleomycin; bortezomib; BSI-201; busulfan; busulfan; calusterone; capecitabine; carboplatin; carfilozib; carmustine; carmustine; celecoxib; cetuximab; chlorambucil; cisplatin; cladribine; clofarabine; cyclophosphamide; cytarabine; cytarabine liposomal; dacarbazine; dactinomycin; darbepoetin alfa; dasatinib; daunorubicin liposomal; daunorubicin; decitabine; denileukin; dexrazoxane; docetaxel; doxorubicin; doxorubicin liposomal; dromostanolone propionate; epirubicin; epoetin alfa; erlotinib; estramustine; etoposide phosphate; etoposide; exemestane; filgrastim; floxuridine; fludarabine; fluorouracil; fulvestrant; gefitinib; gemcitabine; gemtuzumab ozogamicin; goserelin acetate; histrelin acetate; hydroxyurea; Ibritumomab tiuxetan; idarubicin; ifosfamide; imatinib mesylate; interferon alfa 2a; Interferon alfa-2b; irinotecan; lenalidomide; letrozole; leucovorin; leuprolide Acetate; levamisole; lomustine; meclorethamine; megestrol acetate; melphalan; mercaptopurine; methotrexate; methoxsalen; mitomycin C; mitomycin C; mitotane; mitoxantrone; nandrolone phenpropionate; nelarabine; NPI-0052; nofetumomab; oprelvekin; oxaliplatin; paclitaxel; paclitaxel protein-bound particles; palifermin; pamidronate; panitumumab; pegademase; pegaspargase; pegfilgrastim; pemetrexed disodium; pentostatin; pipobroman; plicamycin, mithramycin; porfimer sodium; procarbazine; quinacrine; RAD001; rasburicase; rituximab; sargramostim; Sargramostim; sorafenib; streptozocin; sunitinib malate; tamoxifen; temozolomide; teniposide; testolactone; thalidomide; thioguanine; thiotepa; topotecan; toremifene; tositumomab; tositumomab/I-131 tositumomab; trastuzumab; tretinoin; uracil Mustard; valrubicin; vinblastine; vincristine; vinorelbine; vorinostat; zoledronate; and zoledronic acid.

### FIGURES

FIG. 1 exemplifies effects of administering a combination of pazopanib+abexinostat (PCI-24781 to 786-O human kidney carcinoma cells. Effects of the combination were visualized by measuring alamarBlue.

FIG. 2 exemplifies effects of administering a combination of pazopanib+abexinostat (PCI-24781 to U2-OS osteosarcoma cells. Effects of the combination were visualized by measuring alamarBlue.

### DETAILED DESCRIPTION

Antiangiogenic agents are commonly used in the treatment of various cancers. A common problem associated with antiangiogenic agents is increasing resistance to the agents by tumor cells during treatment. Pazopanib, an antiangiogenic agent, is a tyrosine kinase inhibitor. Resistance to pazopanib often develops during cancer treatment, decreasing the efficacy of pazopanib and ultimately denying patients use of a potentially life-saving medication. There exists a need for new treatment paradigms that decrease or reduce the effects of resistance to antiangiogenic agents such as pazopanib.

HDAC inhibitors produce various epigenetic modifications to the tumor cell genome. These modification may result in increased efficacy of any chemotherapeutic agents co-administered with an HDAC inhibitor. For example, HDAC inhibitors increase accessibility of DNA to various chemotherapeutic agents and therefore increase the cytotoxicity of the chemotherapeutics. N-hydroxy-4-{2-[3-(N,N-dimethylaminomethyl)benzofuran-2-ylcarbonylamino]ethoxy}-benzamide (also known as PCI-24781 or abexinostat) is a hydroxamate-based HDAC inhibitor for use in the treatment of cancer in a human.

Disclosed herein, in certain embodiments, are methods of increasing the effectiveness of an antiangiogenic agent in an individual in need thereof, comprising co-administering to the individual (a) a cycle of abexinostat, or a salt thereof; and (b) an antiangiogenic agent. In some embodiments, the antiangiogenic agent is pazopanib or a salt thereof. In some embodiments, the method reduces resistance to the antiangiogenic agent; delays the development of resistance to the antiangiogenic agent; delays the onset of the cancer becoming refractory to the antiangiogenic agent; prolongs the usefulness of the antiangiogenic agent; allows use of the antiangiogenic agent in the treatment of cancers that generally develop, or have developed, resistance to the antiangiogenic agent; increases patient response to the antiangiogenic agent; increases cellular response to the antiangiogenic agent; decreases the effective dosage of the antiangiogenic agent; or any combination thereof.

Disclosed herein, in certain embodiments, are methods of increasing the effectiveness of pazopanib, or a salt thereof, in an individual in need thereof, comprising co-administering to the individual (a) a cycle of abexinostat, or a salt thereof; and (b) pazopanib, or a salt thereof. In some embodiments, the method reduces resistance to pazopanib, or a salt thereof; delays the development of resistance to pazopanib, or a salt thereof; delays the onset of the cancer becoming refractory to pazopanib, or a salt thereof; prolongs the usefulness of pazopanib, or a salt thereof; allows use of pazopanib, or a salt thereof, in the treatment of cancers that generally develop, or have developed, resistance to pazopanib, or a salt thereof; increases patient response to pazopanib, or a salt thereof; increases cellular response to pazopanib, or a salt thereof; decreases the effective dosage of pazopanib, or a salt thereof; or any combination thereof.

Additionally disclosed herein, in certain embodiments, are methods of treating cancer comprising administering (a) a cycle of abexinostate, or a salt thereof; and (b) an antiangiogenic agent. In some embodiments, the antiangiogenic agent is pazopanib, or a salt thereof. In some embodiments, the method reduces resistance to the antiangiogenic agent; delays the development of resistance to the antiangiogenic agent; delays the onset of the cancer becoming refractory to the antiangiogenic agent; prolongs the usefulness of the antiangiogenic agent; allows use of the antiangiogenic agent in the treatment of cancers that generally develop, or have developed, resistance to the antiangiogenic agent; increases patient response to the antiangiogenic agent; increases cellular response to the antiangiogenic agent; decreases the effective dosage of the antiangiogenic agent; or any combination thereof.

Further disclosed herein, in certain embodiments, are methods of treating cancer comprising administering (a) a cycle of abexinostate, or a salt thereof; and (b) pazopanib, or a salt thereof. In some embodiments, the method reduces resistance to pazopanib, or a salt thereof; delays the development of resistance to pazopanib, or a salt thereof; delays the onset of the cancer becoming refractory to pazopanib, or a salt thereof; prolongs the usefulness of pazopanib, or a salt thereof; allows use of pazopanib, or a salt thereof, in the treatment of cancers that generally develop, or have developed, resistance to pazopanib, or a salt thereof; increases patient response to pazopanib, or a salt thereof; increases cellular response to pazopanib, or a salt thereof; decreases the effective dosage of pazopanib, or a salt thereof; or any combination thereof.

### CERTAIN TERMINOLOGY

The term “pharmaceutical composition” refers to a mixture of an active agent (or ingredient) with other inactive chemical components, such as carriers, stabilizers, diluents, dispersing agents, suspending agents, thickening agents, coatings and/or excipients. The pharmaceutical composition facilitates administration of the compound to a human. In one aspect, the active agent is an HDAC inhibitor (e.g. abexinostat). In one aspect, the active agent is the HCl salt of abexinostat.

“Controlled release” as used herein refers to any release profile that is not entirely immediate release.

“Bioavailability” refers to the percentage of the weight of an HDAC inhibitor (e.g. abexinostat), or a pharmaceutically acceptable salt, dosed that is delivered into the general circulation of the animal or human being studied. The total exposure (AUC_{(0-∞)}) of a drug when administered intravenously is usually defined as 100% Bioavailable (F %). “Oral bioavailability” refers to the extent to which an HDAC inhibitor (e.g. abexinostat), or a pharmaceutically acceptable salt, is absorbed into the general circulation when the pharmaceutical composition is taken orally as compared to intravenous injection.

“Blood plasma concentration” refers to the concentration an HDAC inhibitor (e.g. abexinostat), or a pharmaceutically acceptable salt, in the plasma component of blood of a subject. It is understood that the plasma concentration of an HDAC inhibitor (e.g. abexinostat), or a pharmaceutically acceptable salt, may vary significantly between subjects, due to variability with respect to metabolism and/or interactions with other therapeutic agents. In one aspect, the blood plasma concentration of an HDAC inhibitor (e.g. abexinostat), or a pharmaceutically acceptable salt, varies from subject to subject. Likewise, values such as maximum plasma concentration (Cₘₐₓ) or time to reach maximum plasma concentration (Tₘₐₓ), or total area under the plasma concentration time curve (AUC_{(0-∞)}) vary from subject to subject. Due to this variability, in one embodiment, the amount necessary to constitute “a therapeutically effective amount” of an HDAC inhibitor (e.g. abexinostat), or a pharmaceutically acceptable salt, varies from subject to subject.

“Effective plasma concentrations” of an HDAC inhibitor refers to amounts of the HDAC inhibitor in the plasma that result in exposure levels that are effective for treating a cancer.

“Drug absorption” or “absorption” typically refers to the process of movement of drug from site of administration of a drug across a barrier into a blood vessel or the site of action, e.g., a drug moving from the gastrointestinal tract into the portal vein or lymphatic system.

A “measurable serum concentration” or “measurable plasma concentration” describes the blood serum or blood plasma concentration, typically measured in mg, μg, or ng of therapeutic agent per ml, dl, or l of blood serum, absorbed into the bloodstream after administration. As used herein, measurable plasma concentrations are typically measured in ng/ml or μg/ml.

“Pharmacodynamics” refers to the factors which determine the biologic response observed relative to the concentration of drug at a site of action.

“Pharmacokinetics” refers to the factors which determine the attainment and maintenance of the appropriate concentration of drug at a site of action.

“Drug holiday” means temporarily reducing or temporarily suspending administration of a drug for a certain length of time. The length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. In other embodiments, the dose reduction during a drug holiday is from about 10% to about 100%, including by way of example only about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%.

“Fast mode” or “intergestive” is a physiological state where the stomach exhibits a cyclic activity called the interdigestive migrating motor complex (IMMC). The cyclic activity occurs in four phases: Phase I is the most quiescent, lasts 45 to 60 minutes, and develops few or no contractions; Phase II is marked by the incidence of irregular intermittent sweeping contractions that gradually increase in magnitude; Phase III, which lasts 5 to 15 minutes, is marked by the appearance of intense bursts of peristaltic waves involving both the stomach and the small bowel; and Phase IV is a transition period of decreasing activity which lasts until the next cycle begins. The total cycle time is approximately 90 minutes, and thus, powerful peristaltic waves sweep out the contents of the stomach every 90 minutes during the interdigestive mode. The IMMC may function as an intestinal housekeeper, sweeping swallowed saliva, gastric secretions, and debris to the small intestine and colon, preparing the upper tract for the next meal while preventing bacterial overgrowth. Pancreatic exocrine secretion of pancreatic peptide and motilin also cycle in synchrony with these motor patterns.

“Fed mode” or “postprandial” is a physiological state induced by food ingestion. It begins with changes to the motor pattern of the upper GI tract, the change occurring over a period of 30 seconds to one minute. The stomach generates 3-4 continuous and regular contractions per minute, similar to those of the interdigestive mode but of about half the amplitude. The change occurs almost simultaneously at all sites of the GI tract, before the stomach contents have reached the distal small intestine. Liquids and small particles flow continuously from the stomach into the intestine. Contractions of the stomach result in a sieving process that allows liquids and small particles to pass through a partially open pylorus. Indigestible particles greater than the size of the pylorus are retropelled and retained in the stomach. Particles exceeding about 1 cm in size are thus retained in the stomach for approximately 4 to 6 hours.

As used herein, increasing the effectiveness of an active agent (for example, an antiangiogenic agent, more specifically, pazopanib) includes reducing resistance to the active agent, delaying the development of resistance to the active agent, delaying the onset of the cancer becoming refractory to the active agent, prolonging the usefulness of the active agent, allowing use of the active agent in the treatment of cancers that generally develop, or have developed, resistance to the active agent, increasing patient response to the active agent, increasing cellular response to the active agent, decreasing the effective dosage of the active agent, or any combination thereof.

### Abexinostat

Abexinostat (or, PCI-24781) is a hydroxamate-based HDAC inhibitor. Abexinostat has the chemical name 3-[(dimethylamino)methyl]-N-{2-[4-(hydroxycarbamoyl)phenoxy]ethyl}-1-benzofuran-2-carboxamide.

Disclosed herein, in certain embodiments, are methods of increasing the effectiveness of an antiangiogenic agent in an individual in need thereof, comprising co-administering to the individual (a) a cycle of abexinostat, or a salt thereof; and (b) an antiangiogenic agent. In some embodiments, the antiangiogenic agent is pazopanib or a salt thereof. In some embodiments, the method reduces resistance to the antiangiogenic agent; delays the development of resistance to the antiangiogenic agent; delays the onset of the cancer becoming refractory to the antiangiogenic agent; prolongs the usefulness of the antiangiogenic agent; allows use of the antiangiogenic agent in the treatment of cancers that generally develop, or have developed, resistance to the antiangiogenic agent; increases patient response to the antiangiogenic agent; increases cellular response to the antiangiogenic agent; decreases the effective dosage of the antiangiogenic agent; or any combination thereof.

Disclosed herein, in certain embodiments, are methods of increasing the effectiveness of pazopanib, or a salt thereof, in an individual in need thereof, comprising co-administering to the individual (a) a cycle of abexinostat, or a salt thereof; and (b) pazopanib, or a salt thereof. In some embodiments, the method reduces resistance to pazopanib, or a salt thereof; delays the development of resistance to pazopanib, or a salt thereof; delays the onset of the cancer becoming refractory to pazopanib, or a salt thereof; prolongs the usefulness of pazopanib, or a salt thereof; allows use of pazopanib, or a salt thereof, in the treatment of cancers that generally develop, or have developed, resistance to pazopanib, or a salt thereof; increases patient response to pazopanib, or a salt thereof; increases cellular response to pazopanib, or a salt thereof; decreases the effective dosage of pazopanib, or a salt thereof; or any combination thereof.

Additionally disclosed herein, in certain embodiments, are methods of treating cancer comprising administering (a) a cycle of abexinostate, or a salt thereof; and (b) an antiangiogenic agent. In some embodiments, the antiangiogenic agent is pazopanib, or a salt thereof. In some embodiments, the method reduces resistance to the antiangiogenic agent; delays the development of resistance to the antiangiogenic agent; delays the onset of the cancer becoming refractory to the antiangiogenic agent; prolongs the usefulness of the antiangiogenic agent; allows use of the antiangiogenic agent in the treatment of cancers that generally develop, or have developed, resistance to the antiangiogenic agent; increases patient response to the antiangiogenic agent; increases cellular response to the antiangiogenic agent; decreases the effective dosage of the antiangiogenic agent; or any combination thereof.

Further disclosed herein, in certain embodiments, are methods of treating cancer comprising administering (a) a cycle of abexinostate, or a salt thereof; and (b) pazopanib, or a salt thereof. In some embodiments, the method reduces resistance to pazopanib, or a salt thereof; delays the development of resistance to pazopanib, or a salt thereof; delays the onset of the cancer becoming refractory to pazopanib, or a salt thereof; prolongs the usefulness of pazopanib, or a salt thereof; allows use of pazopanib, or a salt thereof, in the treatment of cancers that generally develop, or have developed, resistance to pazopanib, or a salt thereof; increases patient response to pazopanib, or a salt thereof; increases cellular response to pazopanib, or a salt thereof; decreases the effective dosage of pazopanib, or a salt thereof; or any combination thereof.

Cancers may result fromgenetic defects, such as a gene mutations and deletions and chromosomal abnormalities, that result in the loss of function of tumor suppressor genes and/or gain of function or hyperactivation of oncogenes.

Cancers are often characterized by genome-wide changes in gene expression within the tumor. These changes enhance the ability of a tumor to progress through the cell cycle, avoid apoptosis, or become resistant to chemotherapy. HDAC inhibitors have been shown to reverse several of these changes, and restore a pattern more like that of a normal cell.

The human genome consists of a complex network of genes which are turned on or off depending on the needs of the cell. One of the ways in which genes are turned on or off is by means of chemical modification of histone proteins. Histone proteins are structural components of chromosomes, and form a scaffold upon which DNA, the genetic material, is arranged. A well studied histone modification is acetylation and deacetylation, modifications that are catalyzed by a family of enzymes known as histone acetyl transferases and histone deacetylases.

Inhibition of HDAC enzymes by abexinostat tips the balance in favor of the acetylated state, a state that allows transcription to occur, which can be thought of as turning a gene “on”. When a cell is treated with abexinostat, waves of previously silenced genes are initially turned on. Some of these genes are regulators themselves, and will activate or repress the expression of still other genes. The result is an orchestra of changes to gene expression: some genes being turned on, while others are kept in the off state.

Following chemotherapy and/or radiation treatment, some patient's tumors may turn on certain genes as a strategy by the tumor to adapt to the therapy and become resistant to cell death. One example of a genetic change that occurs in many cancers is the activation of the DNA repair gene RAD51. In response to treatment with DNA-damaging chemotherapy or radiation, tumors will often turn on DNA repair genes (including RAD51) as an adaptive strategy to help the tumor repair the DNA damage done by these agents. In pre-clinical models, abexinostat was able to turn off RAD51 (and other DNA repair genes), effectively blocking the ability of the tumor to repair its damaged DNA, sensitizing the tumor to chemotherapy and radiation.

In preclinical studies abexinostat and salts thereof (e.g., abexinostat HCl) have been found to have anticancer activities with remarkable tumor specificity. These early studies provided important information about the in vitro and in vivo activities of abexinostat and salts thereof (e.g., abexinostat HCl) and determined the molecular mechanism underlying the anticancer effects.

In vitro: abexinostat and salts thereof (e.g., abexinostat (or a salt thereof; e.g., abexinostat HCl) HCl) are active against many tumor cell lines and is efficacious in mouse models of lung, colon, prostate, pancreatic and brain tumors.

Ex vivo: abexinostat and salts thereof (e.g., abexinostat HCl) are active in primary human tumors from patients with colon, ovarian, lung and many hematological cancers.

Extensive safety and toxicology studies have been completed in multiple animal species. The mechanism of action of abexinostat and salts thereof (e.g., abexinostat HCl) have been studied, and involves a multi-pronged attack on tumor cells: upregulation of p21 and other tumor suppressors and cell cycle genes; induction of reactive oxygen species and attenuation of anti-oxidant pathways; alterations in calcium homeostasis and increased ER stress; downregulation of DNA repair pathways and increased DNA damage; direct induction of apoptosis via death receptors and activation of caspases.

In clinical trials involving humans with cancer, abexinostat in solution form was administered at 2 mg/kg as a single oral dose and as multiple 2-hour IV infusion doses. Systemic exposure measured as AUC_{0-∞} for IV and oral dosing was 5.9 μM*hr and 1.45 μM*hr, respectively, indicating an oral bioavailability of about 27% in humans.

### Treatment Regimen

Disclosed herein, in certain embodiments, are methods of increasing the effectiveness of an antiangiogenic agent in an individual in need thereof, comprising co-administering to the individual (a) a cycle of abexinostat, or a salt thereof; and (b) an antiangiogenic agent. In some embodiments, the antiangiogenic agent is pazopanib or a salt thereof. In some embodiments, the method reduces resistance to the antiangiogenic agent; delays the development of resistance to the antiangiogenic agent; delays the onset of the cancer becoming refractory to the antiangiogenic agent; prolongs the usefulness of the antiangiogenic agent; allows use of the antiangiogenic agent in the treatment of cancers that generally develop, or have developed, resistance to the antiangiogenic agent; increases patient response to the antiangiogenic agent; increases cellular response to the antiangiogenic agent; decreases the effective dosage of the antiangiogenic agent; or any combination thereof.

Disclosed herein, in certain embodiments, are methods of increasing the effectiveness of pazopanib, or a salt thereof, in an individual in need thereof, comprising co-administering to the individual (a) a cycle of abexinostat, or a salt thereof; and (b) pazopanib, or a salt thereof. In some embodiments, the method reduces resistance to pazopanib, or a salt thereof; delays the development of resistance to pazopanib, or a salt thereof; delays the onset of the cancer becoming refractory to pazopanib, or a salt thereof; prolongs the usefulness of pazopanib, or a salt thereof; allows use of pazopanib, or a salt thereof, in the treatment of cancers that generally develop, or have developed, resistance to pazopanib, or a salt thereof; increases patient response to pazopanib, or a salt thereof; increases cellular response to pazopanib, or a salt thereof; decreases the effective dosage of pazopanib, or a salt thereof; or any combination thereof.

Additionally disclosed herein, in certain embodiments, are methods of treating cancer comprising administering (a) a cycle of abexinostate, or a salt thereof; and (b) an antiangiogenic agent. In some embodiments, the antiangiogenic agent is pazopanib, or a salt thereof. In some embodiments, the method reduces resistance to the antiangiogenic agent; delays the development of resistance to the antiangiogenic agent; delays the onset of the cancer becoming refractory to the antiangiogenic agent; prolongs the usefulness of the antiangiogenic agent; allows use of the antiangiogenic agent in the treatment of cancers that generally develop, or have developed, resistance to the antiangiogenic agent; increases patient response to the antiangiogenic agent; increases cellular response to the antiangiogenic agent; decreases the effective dosage of the antiangiogenic agent; or any combination thereof.

Further disclosed herein, in certain embodiments, are methods of treating cancer comprising administering (a) a cycle of abexinostate, or a salt thereof; and (b) pazopanib, or a salt thereof. In some embodiments, the method reduces resistance to pazopanib, or a salt thereof; delays the development of resistance to pazopanib, or a salt thereof; delays the onset of the cancer becoming refractory to pazopanib, or a salt thereof; prolongs the usefulness of pazopanib, or a salt thereof; allows use of pazopanib, or a salt thereof, in the treatment of cancers that generally develop, or have developed, resistance to pazopanib, or a salt thereof; increases patient response to pazopanib, or a salt thereof; increases cellular response to pazopanib, or a salt thereof; decreases the effective dosage of pazopanib, or a salt thereof; or any combination thereof.

In some embodiments, the cancer is a hematological cancer, solid tumor or a sarcoma.

In some embodiments, the cancer is a sarcoma. In some embodiments, the cancer is soft tissue sarcoma.

In some embodiments, the cancer is selected from a: breast cancer, colon cancer, colorectal cancer, non-small cell lung cancer, small-cell lung cancer, liver cancer, ovarian cancer, prostate cancer, uterine cervix cancer, urinary bladder cancer, gastric cancer, gastrointestinal stromal tumor, pancreatic cancer, germ cell tumor, mast cell tumor, neuroblastoma, mastocytosis, testicular cancer, glioblastoma, astrocytoma, B cell lymphoma, T cell lymphoma, Hodgkin's lymphoma, non-Hodgkin's lymphoma, melanoma, myeloma, acute myelocytic leukemia (AML), acute lymphocytic leukemia (ALL), myelodysplastic syndrome, chronic myelogenous leukemia, and renal cell carcinoma.

In some embodiments, the cancer is selected from: breast cancer, colon cancer, colorectal carcinomas, non-small cell lung cancer, liver cancer, ovarian cancer, uterine cervix cancer, gastric carcinoma, pancreatic cancer, glioblastomas, B cell lymphoma, T cell lymphoma, Hodgkin's lymphoma, non-Hodgkin's lymphoma, myeloma, myelodysplastic syndrome (MDS), and renal cell carcinoma. In some embodiments, the cancer is renal cell carcinoma or ovarian cancer.

In some embodiments of the methods disclosed herein, an HDAC inhibitor (e.g., abexinostat or a salt thereof such as abexinostat HCl) and pazopanib (or a salt thereof; e.g., pazopanib HCl) are administered in one dosage form (e.g., one oral dosage form). In some embodiments of the methods disclosed herein, an HDAC inhibitor (e.g., abexinostat or a salt thereof such as abexinostat HCl) and pazopanib (or a salt thereof; e.g., pazopanib HCl) are administered separately (i.e., in separate oral dosage forms). Where an HDAC inhibitor (e.g., abexinostat or a salt thereof such as abexinostat HCl) and pazopanib (or a salt thereof; e.g., pazopanib HCl) are administered separately, they are administered concurrently or sequentially. In some embodiments, an HDAC inhibitor (e.g., abexinostat or a salt thereof such as abexinostat HCl) and pazopanib (or a salt thereof; e.g., pazopanib HCl), are administered separately and sequentially. In some embodiments, an HDAC inhibitor (e.g., abexinostat or a salt thereof such as abexinostat HCl) and pazopanib (or a salt thereof; e.g., pazopanib HCl) are administered separately and concurrently.

In some embodiments of the methods disclosed herein, abexinostat (or a salt thereof; e.g., abexinostat HCl), and pazopanib (or a salt thereof; e.g., pazopanib HCl) are administered in one dosage form (e.g., one oral dosage form). In some embodiments of the methods disclosed herein, abexinostat (or a salt thereof; e.g., abexinostat HCl), and pazopanib (or a salt thereof; e.g., pazopanib HCl) are administered separately (i.e., in separate oral dosage forms). Where abexinostat (or a salt thereof; e.g., abexinostat HCl), and pazopanib (or a salt thereof; e.g., pazopanib HCl) are administered separately, they are administered concurrently or sequentially. In some embodiments, abexinostat (or a salt thereof; e.g., abexinostat HCl), and pazopanib (or a salt thereof; e.g., pazopanib HCl), are administered separately and sequentially. In some embodiments, abexinostat (or a salt thereof; e.g., abexinostat HCl), and pazopanib (or a salt thereof; e.g., pazopanib HCl) are administered separately and concurrently.

In some embodiments of the methods disclosed herein, the HDAC inhibitor (e.g., abexinostat or a salt thereof such as abexinostat HCl) and/or pazopanib (or a salt thereof; e.g., pazopanib HCl) are administered by immediate release dosage forms. In some embodiments of the methods disclosed herein, the HDAC inhibitor (e.g., abexinostat or a salt thereof such as abexinostat HCl) and/or pazopanib (or a salt thereof; e.g., pazopanib HCl), are administered by controlled release dosage forms. In some embodiments, the HDAC inhibitor (e.g., abexinostat or a salt thereof such as abexinostat HCl) is administered by a controlled release dosage form and pazopanib, or a salt of pazopanib (e.g., pazopanib HCl), is administered by an immediate release dosage form.

In some embodiments of the methods disclosed herein, abexinostat (or a salt thereof; e.g., abexinostat HCl), and/or pazopanib (or a salt thereof; e.g., pazopanib HCl) are administered by immediate release dosage forms. In some embodiments of the methods disclosed herein, abexinostat (or a salt thereof; e.g., abexinostat HCl), and/or pazopanib (or a salt thereof; e.g., pazopanib HCl), are administered by controlled release dosage forms. In some embodiments, abexinostat (or a salt thereof; e.g., abexinostat HCl) is administered by a controlled release dosage form and pazopanib, or a salt of pazopanib (e.g., pazopanib HCl), is administered by an immediate release dosage form.

In some embodiments, the HDAC inhibitor (e.g., abexinostat or a salt thereof such as abexinostat HCl) and/or pazopanib (or a salt thereof; e.g., pazopanib HCl) are administered orally (e.g., by capsules or tablets). In some embodiments, the HDAC inhibitor (e.g., abexinostat or a salt thereof such as abexinostat HCl) is administered orally (e.g., by capsules or tablets). In some embodiments, pazopanib (or a salt thereof; e.g., pazopanib HCl) is administered orally (e.g., by capsules or tablets).

In some embodiments, abexinostat (or a salt thereof; e.g., abexinostat HCl), and/or pazopanib (or a salt thereof; e.g., pazopanib HCl) are administered orally (e.g., by capsules or tablets). In some embodiments, abexinostat (or a salt thereof; e.g., abexinostat HCl) is administered orally (e.g., by capsules or tablets). In some embodiments, pazopanib (or a salt thereof; e.g., pazopanib HCl) is administered orally (e.g., by capsules or tablets).

In some embodiments, the HDAC inhibitor (e.g., abexinostat or a salt thereof such as abexinostat HCl) and/or pazopanib (or a salt thereof; e.g., pazopanib HCl) are administered intravenously. In some embodiments, the HDAC inhibitor (e.g., abexinostat or a salt thereof such as abexinostat HCl) is administered intravenously. In some embodiments, pazopanib (or a salt thereof; e.g., pazopanib HCl) is administered intravenously.

In some embodiments, abexinostat (or a salt thereof; e.g., abexinostat HCl), and/or pazopanib (or a salt thereof; e.g., pazopanib HCl) are administered intravenously. In some embodiments, abexinostat (or a salt thereof; e.g., abexinostat HCl) is administered intravenously. In some embodiments, pazopanib (or a salt thereof; e.g., pazopanib HCl) is administered intravenously.

In some embodiments of the methods disclosed herein, the HDAC inhibitor (e.g., abexinostat or a salt thereof such as abexinostat HCl) in fast mode. In some embodiments of the methods disclosed herein, pazopanib (or a salt thereof) is administered in fast mode. In some embodiments, the HDAC inhibitor (e.g., abexinostat or a salt thereof such as abexinostat HCl) and pazopanib (or a salt thereof) are administered in fast mode.

In some embodiments of the methods disclosed herein, abexinostat (or a salt thereof) is administered in fast mode. In some embodiments of the methods disclosed herein, pazopanib (or a salt thereof) is administered in fast mode. In some embodiments, abexinostat (or a salt thereof), and pazopanib (or a salt thereof) are administered in fast mode.

In some embodiments of the methods disclosed herein, the HDAC inhibitor (e.g., abexinostat or a salt thereof such as abexinostat HCl) is administered at least about one hour before a meal or at least about 2 hours after a meal. In some embodiments of the methods disclosed herein, pazopanib (or a salt thereof) is administered at least about one hour before a meal or at least about 2 hours after a meal. In some embodiments, the HDAC inhibitor (e.g., abexinostat or a salt thereof such as abexinostat HCl) and pazopanib (or a salt thereof) are administered at least about one hour before a meal or at least about 2 hours after a meal.

In some embodiments of the methods disclosed herein, abexinostat (or a salt thereof) is administered at least about one hour before a meal or at least about 2 hours after a meal. In some embodiments of the methods disclosed herein, pazopanib (or a salt thereof) is administered at least about one hour before a meal or at least about 2 hours after a meal. In some embodiments, abexinostat (or a salt thereof), and pazopanib (or a salt thereof) are administered at least about one hour before a meal or at least about 2 hours after a meal

In some embodiments, the methods disclosed herein comprise administering between about 30 mg/m2 and about 75 mg/m2 of the HDAC inhibitor (e.g., abexinostat or a salt thereof such as abexinostat HCl) BID. In some embodiments, the methods disclosed herein comprise administering between about 400 mg and about 800 mg of pazopanib (or a salt thereof). In some embodiments, the methods disclosed herein comprise administering between about 30 mg/m2 and about 75 mg/m2 of the HDAC inhibitor (e.g., abexinostat or a salt thereof such as abexinostat HCl) BID, and about 200 mg to about 800 mg of pazopanib (or a salt thereof). In some embodiments, the methods disclosed herein comprise administering between about 30 mg/m2 and about 75 mg/m2 of the HDAC inhibitor (e.g., abexinostat or a salt thereof such as abexinostat HCl) BID, and about 216.7 mg to about 866.8 mg of pazopanib HCl.

In some embodiments, the methods disclosed herein comprise administering between about 30 mg/m2 and about 75 mg/m2 of abexinostat (or a salt thereof) BID. In some embodiments, the methods disclosed herein comprise administering between about 400 mg and about 800 mg of pazopanib (or a salt thereof). In some embodiments, the methods disclosed herein comprise administering between about 30 mg/m2 and about 75 mg/m2 of abexinostat (or a salt thereof) BID, and about 200 mg to about 800 mg of pazopanib (or a salt thereof). In some embodiments, the methods disclosed herein comprise administering between about 30 mg/m2 and about 75 mg/m2 of abexinostat (or a salt thereof) BID, and about 216.7 mg to about 866.8 mg of pazopanib HCl.

In some embodiments, the methods disclosed herein comprise administering between about 30 mg/m2 and about 75 mg/m2 of abexinostat (or a salt thereof) BID for 5 days, followed by 2 days without administration of abexinostat (or a salt thereof). In some embodiments, the methods disclosed herein comprise administering between about 400 mg and about 800 mg of pazopanib (or a salt thereof). In some embodiments, the methods disclosed herein comprise administering (a) between about 30 mg/m2 and about 75 mg/m2 of abexinostat (or a salt thereof) BID for 5 days, followed by 2 days without administration of abexinostat (or a salt thereof), and (b) about 200 mg to about 800 mg of pazopanib (or a salt thereof). In some embodiments, the methods disclosed herein comprise administering (a) between about 30 mg/m2 and about 75 mg/m2 of abexinostat (or a salt thereof) BID for 5 days, followed by 2 days without administration of abexinostat (or a salt thereof), and (b) about 216.7 mg to about 866.8 mg of pazopanib HCl.

In some embodiments, the methods disclosed herein are continued until the cancer is in remission. In some embodiments, the methods disclosed herein are continued until disease progression, unacceptable toxicity, or individual choice. In some embodiments, the methods disclosed herein are continued chronically.

### Abexinostat

In some embodiments, the cycle of abexinostat (or a salt thereof; e.g., abexinostat HCl) is 1 to 14 consecutive days, 2 to 14 consecutive days, 3 to 14 consecutive days, 4 to 14 consecutive days, 5 to 14 consecutive days, 6 to 14 consecutive days, 7 to 14 consecutive days, 8 to 14 consecutive days, 9 to 14 consecutive days, 10 to 14 consecutive days, 11 to 14 consecutive days, 12 to 14 consecutive days, or 13 to 14 consecutive days. In some embodiments, the cycle of abexinostat (or a salt thereof; e.g., abexinostat HCl) is 1 to 14 consecutive days. In some embodiments, the cycle of abexinostat (or a salt thereof; e.g., abexinostat HCl) is 2 to 14 consecutive days. In some embodiments, the cycle of abexinostat (or a salt thereof; e.g., abexinostat HCl) is 3 to 14 consecutive days. In some embodiments, the cycle of abexinostat (or a salt thereof; e.g., abexinostat HCl) is 4 to 14 consecutive days. In some embodiments, the cycle of abexinostat (or a salt thereof; e.g., abexinostat HCl) is 5 to 14 consecutive days. In some embodiments, the cycle of abexinostat (or a salt thereof; e.g., abexinostat HCl) is 6 to 14 consecutive days. In some embodiments, the cycle of abexinostat (or a salt thereof; e.g., abexinostat HCl) is 7 to 14 consecutive days. In some embodiments, the cycle of abexinostat (or a salt thereof; e.g., abexinostat HCl) is 8 to 14 consecutive days. In some embodiments, the cycle of abexinostat (or a salt thereof; e.g., abexinostat HCl) is 9 to 14 consecutive days. In some embodiments, the cycle of abexinostat (or a salt thereof; e.g., abexinostat HCl) is 10 to 14 consecutive days. In some embodiments, the cycle of abexinostat (or a salt thereof; e.g., abexinostat HCl) is 11 to 14 consecutive days. In some embodiments, the cycle of abexinostat (or a salt thereof; e.g., abexinostat HCl) is 12 to 14 consecutive days. In some embodiments, the cycle of abexinostat (or a salt thereof; e.g., abexinostat HCl) is 13 to 14 consecutive days. In some embodiments, the cycle of abexinostat (or a salt thereof; e.g., abexinostat HCl) is 5 to 9 days. In some embodiments, the cycle of abexinostat (or a salt thereof; e.g., abexinostat HCl) is 6 to 8 days.

In some embodiments, the cycle of abexinostat (or a salt thereof; e.g., abexinostat HCl) is 2 consecutive days, 3 consecutive days, 4 consecutive days, 5 consecutive days, 6 consecutive days, 7 consecutive days, 8 consecutive days, 9 consecutive days, 10 consecutive days, 11 consecutive days, 12 consecutive days, 13 consecutive days, or 14 consecutive days. In some embodiments, the cycle of abexinostat (or a salt thereof; e.g., abexinostat HCl) is 2 consecutive days. In some embodiments, the cycle of abexinostat (or a salt thereof; e.g., abexinostat HCl) is 3 consecutive days. In some embodiments, the cycle of abexinostat (or a salt thereof; e.g., abexinostat HCl) is 4 consecutive days. In some embodiments, the cycle of abexinostat (or a salt thereof; e.g., abexinostat HCl) is 5 consecutive days. In some embodiments, the cycle of abexinostat (or a salt thereof; e.g., abexinostat HCl) is 6 consecutive days. In some embodiments, the cycle of abexinostat (or a salt thereof; e.g., abexinostat HCl) is 7 consecutive days. In some embodiments, the cycle of abexinostat (or a salt thereof; e.g., abexinostat HCl) is 8 consecutive days. In some embodiments, the cycle of abexinostat (or a salt thereof; e.g., abexinostat HCl) is 9 consecutive days. In some embodiments, the cycle of abexinostat (or a salt thereof; e.g., abexinostat HCl) is 10 consecutive days. In some embodiments, the cycle of abexinostat (or a salt thereof; e.g., abexinostat HCl) is 11 consecutive days. In some embodiments, the cycle of abexinostat (or a salt thereof; e.g., abexinostat HCl) is 12 consecutive days. In some embodiments, the cycle of abexinostat (or a salt thereof; e.g., abexinostat HCl) is 13 consecutive days. In some embodiments, the cycle of abexinostat (or a salt thereof; e.g., abexinostat HCl) is 14 consecutive days.

In some embodiments, abexinostat (or a salt thereof; e.g., abexinostat HCl) is administered once per day during a cycle of abexinostat (or a salt thereof; e.g., abexinostat HCl). In some embodiments, abexinostat (or a salt thereof; e.g., abexinostat HCl) is administered twice per day during a cycle of abexinostat (or a salt thereof; e.g., abexinostat HCl). In some embodiments, abexinostat (or a salt thereof; e.g., abexinostat HCl) is administered three times per day during a cycle of abexinostat (or a salt thereof; e.g., abexinostat HCl). In certain instances, twice a day dosing reduces the incidences of thrombocytopenia as compared to three times a day dosing.

In some embodiments, abexinostat (or a salt thereof; e.g., abexinostat HCl) is administered twice per day during a cycle of abexinostat (or a salt thereof; e.g., abexinostat HCl). In some embodiments, each dose of abexinostat (or a salt thereof; e.g., abexinostat HCl) is administered 4 to 8 hours apart. In some embodiments, any of the methods disclosed herein comprise administering a first dose of abexinostat (or a salt thereof; e.g., abexinostat HCl) and a second dose of abexinostat (or a salt thereof; e.g., abexinostat HCl), wherein the first dose and the second dose are administered 4 to 8 hours apart.

In some embodiments, abexinostat (or a salt thereof; e.g., abexinostat HCl) is administered three times per day during a cycle of abexinostat (or a salt thereof; e.g., abexinostat HCl). In some embodiments, each dose of abexinostat (or a salt thereof; e.g., abexinostat HCl) is administered 4 to 8 hours apart. In some embodiments, any of the methods disclosed herein comprise administering a first dose of abexinostat (or a salt thereof; e.g., abexinostat HCl), a second dose of abexinostat (or a salt thereof; e.g., abexinostat HCl) and a third dose of abexinostat (or a salt thereof; e.g., abexinostat HCl), wherein the first dose, the second dose and the third dose are administered 4 to 8 hours apart.

For therapeutic effect, the effective plasma concentration of abexinostat in humans should be maintained for at least 6 consecutive hours, at least 7 consecutive hours, or at least 8 consecutive hours each day on days of dosing. Maintaining the effective plasma concentrations for about 6 consecutive hours to about 8 consecutive hours of abexinostat on days of dosing increases the efficacy of tumor cell growth inhibition and minimizes the incidences of thrombocytopenia.

In some embodiments, the effective plasma concentration of abexinostat in humans is maintained for at least 6 consecutive hours each day on days of dosing. In some embodiments, a dose of abexinostat (or a salt thereof; e.g., abexinostat HCl) is sufficient to maintain an effective plasma concentration of the HDAC inhibitor in the individual for at least about 6 consecutive hours.

In some embodiments, the effective plasma concentration of abexinostat in humans is maintained for at least 7 consecutive hours each day on days of dosing. In some embodiments, a dose of abexinostat (or a salt thereof; e.g., abexinostat HCl) is sufficient to maintain an effective plasma concentration of the HDAC inhibitor in the individual for at least about 7 consecutive hours.

In some embodiments, the effective plasma concentration of abexinostat in humans is maintained for at least 8 consecutive hours each day on days of dosing. In some embodiments, a dose of abexinostat (or a salt thereof; e.g., abexinostat HCl) is sufficient to maintain an effective plasma concentration of the HDAC inhibitor in the individual for at least about 8 consecutive hours.

In some embodiments, the effective plasma concentration of abexinostat in humans is maintained for at least 6 consecutive hours but not exceeding 12, 13, or 14 consecutive hours on days of dosing. Maintaining the effective plasma concentrations for at least 6 consecutive hours but not exceeding 14 consecutive hours of abexinostat on days of dosing increases the efficacy of tumor cell growth inhibition and minimizes the incidences of thrombocytopenia.

The oral bioavailability of abexinostat in humans, administered as immediate release capsules or an oral solution, was determined to be about 27%. A difference in pharmacokinetics was observed in laboratory animals between the fasted state the fed state. Abexinostat appears to be preferentially absorbed in the intestines.

Daily amounts of abexinostat which are administered to humans range from about 10 mg/mm² to about 200 mg/mm². In some embodiments, the daily dose of abexinostat is between about 30 mg/mm² to about 90 mg/mm². In some embodiments, the daily dose of abexinostat is between about 60 mg/mm² to about 150 mg/mm². In some embodiments, the daily dose of abexinostat is about 20 mg/mm², about 30 mg/mm², about 40 mg/mm², about 50 mg/mm², about 60 mg/mm², about 70 mg/mm², about 80 mg/mm², about 90 mg/mm², about 100 mg/mm², about 110 mg/mm², about 120 mg/mm², about 130 mg/mm², about 140 mg/mm², or about 150 mg/mm². In some embodiments, the daily dose of abexinostat is about 20 mg/mm². In some embodiments, the daily dose of abexinostat is about 30 mg/mm². In some embodiments, the daily dose of abexinostat is about 40 mg/mm². In some embodiments, the daily dose of abexinostat is about 50 mg/mm². In some embodiments, the daily dose of abexinostat is about 60 mg/mm². In some embodiments, the daily dose of abexinostat is about 70 mg/mm². In some embodiments, the daily dose of abexinostat is about 80 mg/mm². In some embodiments, the daily dose of abexinostat is about 90 mg/mm². In some embodiments, the daily dose of abexinostat is about 100 mg/mm². In some embodiments, the daily dose of abexinostat is about 110 mg/mm². In some embodiments, the daily dose of abexinostat is about 120 mg/mm². In some embodiments, the daily dose of abexinostat is about 130 mg/mm². In some embodiments, the daily dose of abexinostat is about 140 mg/mm². In some embodiments, the daily dose of abexinostat is about 150 mg/mm².

In some embodiments, the daily dose of abexinostat is between about 40 mg to about 600 mg of abexinostat.

The daily dose of abexinostat (or a salt thereof; e.g., abexinostat HCl) that is administered varies depending upon factors including, by way of non-limiting example, the type of formulation utilized, the type of cancer and its severity, the identity (e.g., weight, age) of the human, and/or the route of administration.

In some embodiments of the methods disclosed herein, abexinostat (or a salt thereof; e.g., abexinostat HCl) is administered by immediate release dosage forms. In some embodiments of the methods disclosed herein, abexinostat (or a salt thereof; e.g., abexinostat HCl) is administered by controlled release dosage forms.

In some embodiments, the dosage form completely releases abexinostat (or a salt thereof) over a period of about 2 hours to about 10 hours after administration.

In some embodiments, abexinostat (or a salt thereof; e.g., abexinostat HCl) is administered orally (e.g., by capsules or tablets). In some embodiments, abexinostat (or a salt thereof; e.g., abexinostat HCl) is administered by an immediate release oral dosage form (e.g., by capsules or tablets). In some embodiments, abexinostat (or a salt thereof; e.g., abexinostat HCl) is administered by a controlled release oral dosage form (e.g., by capsules or tablets).

In some embodiments of the methods disclosed herein, the methods comprise administering a first immediate release oral dosage form comprising abexinostat (or a salt thereof) and a second immediate release oral dosage form comprising abexinostat (or a salt thereof), wherein the second immediate release oral dosage form is administered about 4 to about 8 hours form the first immediate release oral dosage form.

In some embodiments, abexinostat (or a salt thereof; e.g., abexinostat HCl) is administered intravenously.

In some embodiments, abexinostat (or a salt thereof; e.g., abexinostat HCl) is administered when the individual is in fast mode. In some embodiments, abexinostat (or a salt thereof; e.g., abexinostat HCl) is administered at least about 1 hour before a meal. In some embodiments, abexinostat (or a salt thereof; e.g., abexinostat HCl) is administered at least about 2 hours after a meal.

In some embodiments, abexinostat (or a salt thereof) is administered until the cancer is in remission. In some embodiments, abexinostat (or a salt thereof) is administered until disease progression, unacceptable toxicity, or individual choice. In some embodiments, abexinostat (or a salt thereof) is administered chronically.

### Abexinostat Drug Holiday

In certain instances, thrombocytopenia is a side effect observed in humans that receive treatment with HDAC inhibitor compounds. Grade 4 thrombocytopenia typically includes instances when the human has a platelet count less than 25,000 per mm². Thrombocytopenia may be ameliorated or avoided by lowering the daily dose of abexinostat. In some embodiment, a method disclosed herein further comprises an abexinostat (or a salt thereof; e.g., abexinostat HCl) drug holiday following an abexinostat (or a salt thereof; e.g., abexinostat HCl) cycle. In some embodiments, the abexinostat (or a salt thereof; e.g., abexinostat HCl) drug holiday does not compromise the efficacy of an abexinostat (or a salt thereof; e.g., abexinostat HCl) treatment regimen.

In some embodiments, the abexinostat (or a salt thereof; e.g., abexinostat HCl) abexinostat (or a salt thereof; e.g., abexinostat HCl) drug holiday is 1 to 14 consecutive days, 2 to 14 consecutive days, 3 to 14 consecutive days, 4 to 14 consecutive days, 5 to 14 consecutive days, 6 to 14 consecutive days, 7 to 14 consecutive days, 8 to 14 consecutive days, 9 to 14 consecutive days, 10 to 14 consecutive days, 11 to 14 consecutive days, 12 to 14 consecutive days, or 13 to 14 consecutive days. In some embodiments, the abexinostat (or a salt thereof; e.g., abexinostat HCl) abexinostat (or a salt thereof; e.g., abexinostat HCl) drug holiday is 1 to 14 consecutive days. In some embodiments, the abexinostat (or a salt thereof; e.g., abexinostat HCl) abexinostat (or a salt thereof; e.g., abexinostat HCl) drug holiday 2 to 14 consecutive days. In some embodiments, the abexinostat (or a salt thereof; e.g., abexinostat HCl) abexinostat (or a salt thereof; e.g., abexinostat HCl) drug holiday 3 to 14 consecutive days. In some embodiments, the abexinostat (or a salt thereof; e.g., abexinostat HCl) abexinostat (or a salt thereof; e.g., abexinostat HCl) drug holiday 4 to 14 consecutive days. In some embodiments, the abexinostat (or a salt thereof; e.g., abexinostat HCl) abexinostat (or a salt thereof; e.g., abexinostat HCl) drug holiday 5 to 14 consecutive days. In some embodiments, the abexinostat (or a salt thereof; e.g., abexinostat HCl) abexinostat (or a salt thereof; e.g., abexinostat HCl) drug holiday 6 to 14 consecutive days. In some embodiments, the abexinostat (or a salt thereof; e.g., abexinostat HCl) abexinostat (or a salt thereof; e.g., abexinostat HCl) drug holiday 7 to 14 consecutive days. In some embodiments, the abexinostat (or a salt thereof; e.g., abexinostat HCl) abexinostat (or a salt thereof; e.g., abexinostat HCl) drug holiday 8 to 14 consecutive days. In some embodiments, the abexinostat (or a salt thereof; e.g., abexinostat HCl) abexinostat (or a salt thereof; e.g., abexinostat HCl) drug holiday 9 to 14 consecutive days. In some embodiments, the abexinostat (or a salt thereof; e.g., abexinostat HCl) abexinostat (or a salt thereof; e.g., abexinostat HCl) drug holiday 10 to 14 consecutive days. In some embodiments, the abexinostat (or a salt thereof; e.g., abexinostat HCl) abexinostat (or a salt thereof; e.g., abexinostat HCl) drug holiday 11 to 14 consecutive days. In some embodiments, the abexinostat (or a salt thereof; e.g., abexinostat HCl) abexinostat (or a salt thereof; e.g., abexinostat HCl) drug holiday 12 to 14 consecutive days. In some embodiments, the abexinostat (or a salt thereof; e.g., abexinostat HCl) abexinostat (or a salt thereof; e.g., abexinostat HCl) drug holiday 13 to 14 consecutive days.

In some embodiments, the abexinostat (or a salt thereof; e.g., abexinostat HCl) abexinostat (or a salt thereof; e.g., abexinostat HCl) drug holiday is 2 consecutive days, 3 consecutive days, 4 consecutive days, 5 consecutive days, 6 consecutive days, 7 consecutive days, 8 consecutive days, 9 consecutive days, 10 consecutive days, 11 consecutive days, 12 consecutive days, 13 consecutive days, or 14 consecutive days. In some embodiments, the abexinostat (or a salt thereof; e.g., abexinostat HCl) abexinostat (or a salt thereof; e.g., abexinostat HCl) drug holiday is 2 consecutive days. In some embodiments, the abexinostat (or a salt thereof; e.g., abexinostat HCl) abexinostat (or a salt thereof; e.g., abexinostat HCl) drug holiday is 3 consecutive days. In some embodiments, the abexinostat (or a salt thereof; e.g., abexinostat HCl) abexinostat (or a salt thereof; e.g., abexinostat HCl) drug holiday is 4 consecutive days. In some embodiments, the abexinostat (or a salt thereof; e.g., abexinostat HCl) abexinostat (or a salt thereof; e.g., abexinostat HCl) drug holiday is 5 consecutive days. In some embodiments, the abexinostat (or a salt thereof; e.g., abexinostat HCl) abexinostat (or a salt thereof; e.g., abexinostat HCl) drug holiday is 6 consecutive days. In some embodiments, the abexinostat (or a salt thereof; e.g., abexinostat HCl) abexinostat (or a salt thereof; e.g., abexinostat HCl) drug holiday is 7 consecutive days. In some embodiments, the abexinostat (or a salt thereof; e.g., abexinostat HCl) abexinostat (or a salt thereof; e.g., abexinostat HCl) drug holiday is 8 consecutive days. In some embodiments, the abexinostat (or a salt thereof; e.g., abexinostat HCl) abexinostat (or a salt thereof; e.g., abexinostat HCl) drug holiday is 9 consecutive days. In some embodiments, the abexinostat (or a salt thereof; e.g., abexinostat HCl) abexinostat (or a salt thereof; e.g., abexinostat HCl) drug holiday is 10 consecutive days. In some embodiments, the abexinostat (or a salt thereof; e.g., abexinostat HCl) abexinostat (or a salt thereof; e.g., abexinostat HCl) drug holiday is 11 consecutive days. In some embodiments, the abexinostat (or a salt thereof; e.g., abexinostat HCl) abexinostat (or a salt thereof; e.g., abexinostat HCl) drug holiday is 12 consecutive days. In some embodiments, the abexinostat (or a salt thereof; e.g., abexinostat HCl) abexinostat (or a salt thereof; e.g., abexinostat HCl) drug holiday is 13 consecutive days. In some embodiments, the abexinostat (or a salt thereof; e.g., abexinostat HCl) abexinostat (or a salt thereof; e.g., abexinostat HCl) drug holiday is 14 consecutive days.

In some embodiments, the methods disclosed herein comprise 5-9 consecutive days of daily dosing of abexinostat (or a salt thereof; e.g., abexinostat HCl), followed by 5-9 consecutive days without dosing abexinostat (or a salt thereof; e.g., abexinostat HCl). In some embodiments, the methods disclosed herein comprise 5-9 consecutive days of daily dosing of abexinostat (or a salt thereof; e.g., abexinostat HCl), followed by 2-9 consecutive days without dosing abexinostat (or a salt thereof; e.g., abexinostat HCl). In some embodiments, the methods disclosed herein comprise 6-8 consecutive days of daily dosing of abexinostat (or a salt thereof; e.g., abexinostat HCl), followed by 6-8 consecutive days without dosing abexinostat (or a salt thereof; e.g., abexinostat HCl). In some embodiments, the methods disclosed herein comprise 6-8 consecutive days of daily dosing of abexinostat (or a salt thereof; e.g., abexinostat HCl), followed by 2-8 consecutive days without dosing abexinostat (or a salt thereof; e.g., abexinostat HCl).

In some embodiments, the methods disclosed herein comprise 7 consecutive days of daily dosing of abexinostat (or a salt thereof; e.g., abexinostat HCl), followed by 7 consecutive days without dosing abexinostat (or a salt thereof; e.g., abexinostat HCl).

In some embodiments, the methods disclosed herein comprise 5 consecutive days of daily dosing of abexinostat (or a salt thereof; e.g., abexinostat HCl), followed by 2 consecutive days without dosing abexinostat (or a salt thereof; e.g., abexinostat HCl).

### Pazopanib

Pazopanib, 5-[[4-[(2,3-Dimethyl-2H-indazol-6-yl)(methyl)amino]pyrimidin-2-yl]amino]-2-methylbenzenesulfonamide monohydrochloride, is an oral angiogenesis inhibitor targeting the tyrosine kinase activity associated with vascular endothelial growth factor receptor (VEGFR)-1, -2 and -3, platelet-derived growth factor receptor (PDGFR)-α, and PDGFR-β, and stem cell factor receptor (c-KIT).

In some embodiments, pazopanib (or a salt thereof; e.g., pazopanib HCl), is administered to an individual in combination with abexinostat (or a salt thereof; e.g., abexinostat HCl). In some embodiments, pazopanib is administered to an individual in combination with abexinostat (or a salt thereof; e.g., abexinostat HCl). In some embodiments, pazopanib HCl is administered to an individual in combination with abexinostat (or a salt thereof; e.g., abexinostat HCl). In some embodiments, pazopanib HCl is administered to an individual in combination with a salt of abexinostat (e.g., abexinostat HCl).

In some embodiments, pazopanib (or a salt thereof; e.g., pazopanib HCl) is administered to the individual continuously, e.g., without drug holidays. In some embodiments, administration of pazopanib (or a salt thereof; e.g., pazopanib HCl), is not halted on the days that abexinostat is not administered (i.e., during an abexinostat drug holiday). In some embodiments, administration of pazopanib (or a salt thereof; e.g., pazopanib HCl) is halted on the days that abexinostat is not administered (i.e., during an abexinostat drug holiday).

In some embodiments, pazopanib (or a salt thereof; e.g., pazopanib HCl) is administered by an immediate release dosage form. In some embodiments, pazopanib (or a salt thereof; e.g., pazopanib HCl) is administered by a controlled release dosage form.

In some embodiments, pazopanib (or a salt thereof; e.g., pazopanib HCl) is administered orally (e.g., by capsules or tablets). In some embodiments, pazopanib (or a salt thereof; e.g., pazopanib HCl) is administered by an immediate release oral dosage form (e.g., by capsules or tablets). In some embodiments, pazopanib (or a salt thereof; e.g., pazopanib HCl) is administered by a controlled release oral dosage form (e.g., by capsules or tablets).

In some embodiments, pazopanib (or a salt thereof; e.g., pazopanib HCl) is administered intravenously.

In some embodiments, abexinostat (or a salt thereof) is administered until the cancer is in remission. In some embodiments, abexinostat (or a salt thereof) is administered until disease progression, unacceptable toxicity, or individual choice. In some embodiments, abexinostat (or a salt thereof) is administered chronically.

In some embodiments, pazopanib (or a salt thereof; e.g., pazopanib HCl) is administered when the individual is in fast mode. In some embodiments, pazopanib (or a salt thereof; e.g., pazopanib HCl) is administered at least about 1 hour before a meal. In some embodiments, pazopanib (or a salt thereof; e.g., pazopanib HCl) is administered at least about 2 hours after a meal.

In some embodiments, pazopanib (or a salt thereof) is administered once per day, twice per day, three times per day, or four times per day. In some embodiments, pazopanib (or a salt thereof) is administered once per day. In some embodiments, pazopanib (or a salt thereof) is administered twice per day. In some embodiments, pazopanib (or a salt thereof) is administered three times per day. In some embodiments, pazopanib (or a salt thereof) is administered four times per day.

In some embodiments, pazopanib (or a salt thereof) is administered twice per day. In some embodiments, each dose of pazopanib (or a salt thereof) is administered 4 to 8 hours apart. In some embodiments, any of the methods disclosed herein comprise administering a first dose of pazopanib (or a salt thereof) and a second dose of pazopanib (or a salt thereof), wherein the first dose and the second dose are administered 4 to 8 hours apart.

In some embodiments, pazopanib (or a salt thereof) is administered three times per day. In some embodiments, each dose of pazopanib (or a salt thereof) is administered 4 to 8 hours apart. In some embodiments, any of the methods disclosed herein comprise administering a first dose of pazopanib (or a salt thereof), a second dose of pazopanib (or a salt thereof) and a third dose of pazopanib (or a salt thereof), wherein the first dose, the second dose and the third dose are administered 4 to 8 hours apart.

In some embodiments, pazopanib (or a salt thereof) is administered four times per day. In some embodiments, each dose of pazopanib (or a salt thereof) is administered 4 to 8 hours apart. In some embodiments, any of the methods disclosed herein comprise administering a first dose of pazopanib (or a salt thereof), a second dose of pazopanib (or a salt thereof), a third dose of pazopanib (or a salt thereof), and a fourth dose of pazopanib (or a salt thereof), wherein the first dose, the second dose, the third dose and the fourth dose are administered 4 to 8 hours apart.

In some embodiments, the daily dose of pazopanib is about 200 mg to about 800 mg, about 400 mg to about 800 mg, or about 600 mg to about 800 mg. In some embodiments, the daily dose of pazopanib is about 200 mg to about 800 mg. In some embodiments, the daily dose of pazopanib is about 400 mg to about 800 mg. In some embodiments, the daily dose of pazopanib is about 600 mg to about 800 mg.

In some embodiments, the daily dose of pazopanib is about 200 mg, about 400 mg, about 600 mg or about 800 mg. In some embodiments, the daily dose of pazopanib is about 200 mg. In some embodiments, the daily dose of pazopanib is about 400 mg. In some embodiments, the daily dose of pazopanib is about 600 mg. In some embodiments, the daily dose of pazopanib is about 800 mg.

In some embodiments, the daily dose of pazopanib HCl is about 216.7 mg to about 866.8 mg, about 433.4 mg to about 866.8 mg, or about 650.1 mg to about 866.8 mg. In some embodiments, the daily dose of pazopanib HCl is about 216.7 mg to about 866.8 mg. In some embodiments, the daily dose of pazopanib HCl is about 433.4 mg to about 866.8 mg. In some embodiments, the daily dose of pazopanib HCl is about 650.1 mg to about 866.8 mg.

In some embodiments, the daily dose of pazopanib HCl is about 216.7 mg, about 433.4 mg, about 650.1 mg or about 866.8 mg. In some embodiments, the daily dose of pazopanib HCl is about 216.7 mg. In some embodiments, the daily dose of pazopanib HCl is about 433.4 mg. In some embodiments, the daily dose of pazopanib HCl is about 650.1 mg. In some embodiments, the daily dose of pazopanib HCl is about 866.8 mg.

The daily dose of abexinostat (or a salt thereof; e.g., abexinostat HCl) that is administered varies depending upon factors including, by way of non-limiting example, the type of formulation utilized, the type of cancer and its severity, the identity (e.g., weight, age) of the human, and/or the route of administration.

### HDAC Inhibitor Compounds

Disclosed herein, in certain embodiments, are methods of increasing the effectiveness of an antiangiogenic agent in an individual in need thereof, comprising co-administering to the individual (a) a cycle of an HDAC inhibitor, or a salt thereof; and (b) an antiangiogenic agent. In some embodiments, the HDAC inhibitor is abexinostat. In some embodiments, the antiangiogenic agent is pazopanib or a salt thereof. In some embodiments, the method reduces resistance to the antiangiogenic agent; delays the development of resistance to the antiangiogenic agent; delays the onset of the cancer becoming refractory to the antiangiogenic agent; prolongs the usefulness of the antiangiogenic agent; allows use of the antiangiogenic agent in the treatment of cancers that generally develop, or have developed, resistance to the antiangiogenic agent; increases patient response to the antiangiogenic agent; increases cellular response to the antiangiogenic agent; decreases the effective dosage of the antiangiogenic agent; or any combination thereof.

Disclosed herein, in certain embodiments, are methods of increasing the effectiveness of pazopanib, or a salt thereof, in an individual in need thereof, comprising co-administering to the individual (a) a cycle of an HDAC inhibitor, or a salt thereof; and (b) pazopanib, or a salt thereof. In some embodiments, the HDAC inhibitor is abexinostat. In some embodiments, the method reduces resistance to pazopanib, or a salt thereof; delays the development of resistance to pazopanib, or a salt thereof; delays the onset of the cancer becoming refractory to pazopanib, or a salt thereof; prolongs the usefulness of pazopanib, or a salt thereof; allows use of pazopanib, or a salt thereof, in the treatment of cancers that generally develop, or have developed, resistance to pazopanib, or a salt thereof; increases patient response to pazopanib, or a salt thereof; increases cellular response to pazopanib, or a salt thereof; decreases the effective dosage of pazopanib, or a salt thereof; or any combination thereof.

Additionally disclosed herein, in certain embodiments, are methods of treating cancer comprising administering (a) a cycle of an HDAC inhibitor, or a salt thereof; and (b) an antiangiogenic agent. In some embodiments, the HDAC inhibitor is abexinostat. In some embodiments, the antiangiogenic agent is pazopanib, or a salt thereof. In some embodiments, the method reduces resistance to the antiangiogenic agent; delays the development of resistance to the antiangiogenic agent; delays the onset of the cancer becoming refractory to the antiangiogenic agent; prolongs the usefulness of the antiangiogenic agent; allows use of the antiangiogenic agent in the treatment of cancers that generally develop, or have developed, resistance to the antiangiogenic agent; increases patient response to the antiangiogenic agent; increases cellular response to the antiangiogenic agent; decreases the effective dosage of the antiangiogenic agent; or any combination thereof.

Further disclosed herein, in certain embodiments, are methods of treating cancer comprising administering (a) a cycle of an HDAC inhibitor, or a salt thereof; and (b) pazopanib, or a salt thereof. In some embodiments, the HDAC inhibitor is abexinostat. In some embodiments, the method reduces resistance to pazopanib, or a salt thereof; delays the development of resistance to pazopanib, or a salt thereof; delays the onset of the cancer becoming refractory to pazopanib, or a salt thereof; prolongs the usefulness of pazopanib, or a salt thereof; allows use of pazopanib, or a salt thereof, in the treatment of cancers that generally develop, or have developed, resistance to pazopanib, or a salt thereof; increases patient response to pazopanib, or a salt thereof; increases cellular response to pazopanib, or a salt thereof; decreases the effective dosage of pazopanib, or a salt thereof; or any combination thereof.

N-hydroxy-4-{2-[3-(N,N-dimethylaminomethyl)benzofuran-2-ylcarbonylamino]ethoxy}-benzamide (abexinostat) has the following structure:

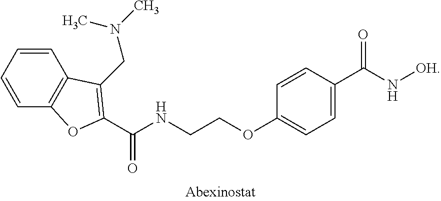 [SMILES: CN(C)Cc1c(C(=O)NCCOc2ccc(C(=O)NO)cc2)oc2ccccc12]

In one aspect, abexinostat is used in the methods disclosed herein as a pharmaceutically acceptable salt. In one aspect, abexinostat is used as the hydrochloride salt.

Additional pharmaceutically acceptable salts of abexinostat include: (a) salts formed when the acidic proton of abexinostat is replaced by a metal ion, such as for example, an alkali metal ion (e.g. lithium, sodium, potassium), an alkaline earth ion (e.g. magnesium, or calcium), or an aluminum ion, or is replaced by an ammonium cation (NH₄⁺); (b) salts formed by reacting abexinostat with a pharmaceutically acceptable organic base, which includes alkylamines, such as ethanolamine, diethanolamine, triethanolamine, tromethamine, N-methylglucamine, dicyclohexylamine, tris(hydroxymethyl)methylamine, and salts with amino acids such as arginine, lysine, and the like; (c) salts formed by reacting abexinostat with a pharmaceutically acceptable acid, which provides acid addition salts. Pharmaceutically acceptable acids include hydrochloric acid, hydrobromic acid, sulfuric acid, nitric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

Additional pharmaceutically acceptable salts include those described in Berge et al., J. Pharm. Sci. 1977, 66, 1-19; and “Handbook of Pharmaceutical Salts, Properties, and Use,” Stah and Wermuth, Ed.; Wiley-VCH and VHCA, Zurich, 2002.

In some embodiments, sites on the aromatic ring portion of compounds described herein that are susceptible to various metabolic reactions are modified such that the various metabolic reactions are reduced, minimized or eliminated. Such modifications include incorporation of appropriate substituents on the aromatic ring structures, such as, by way of example only, halogens, deuterium, and the like. In one aspect, HDAC inhibitor compounds described herein are deuterated at sites susceptible to metabolic reactions.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as, for example, ²H, ³H, ¹³C, ¹⁴C, ¹⁵N, ¹⁸O, ¹⁷O, ³⁵S, ¹⁸F, ³⁶Cl, respectively. Certain isotopically-labeled compounds described herein, for example those into which radioactive isotopes such as ³H and ¹⁴C are incorporated, are useful in drug and/or substrate tissue distribution assays. Further, substitution with isotopes such as deuterium, i.e., ²H, can afford certain therapeutic advantages resulting from greater metabolic stability, for example increased in vivo half-life or reduced dosage requirements.

Other HDAC inhibitor compounds that are contemplated for use in the pharmaceutical compositions, pharmacokinetic strategies, dosing regimens, methods of treatments, and combination therapies include those compounds with the structure of Formula (I):

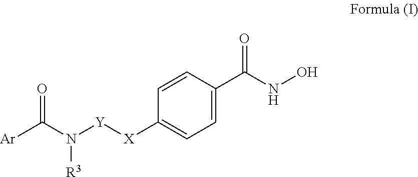

wherein:
X is —O—, —NR²—, or —S(O)ₙ where n is 0, 1, or 2 and R² is hydrogen, —CH₃, —CH₂CH₃;
Y is ethylene, propylene, 1-methylpropylene, 2-methylpropylene, —CH(C₂H₅)CH₂—, —CH(CH(CH₃)₂)CH₂—, and —CH(CH₃)CH₂—;
R³ is hydrogen, —CH₃, or —CH₂CH₃;
Ar is phenyl, naphthyl, quinolinyl, benzofuranyl, benzothienyl, trans phenylCH═CH— or trans (benzofuran-2-yl)CH═CH—, wherein Ar is optionally substituted with one or two substituents independently selected from chloro, fluoro, trifluoromethyl, methyl, ethyl, methoxy, ethoxy, methylenedioxy, —OH, 1-cyclopropylpiperidin-4-yloxy, 1-(2,2,2-trifluoroethyl)piperidin-4-yloxy, N,N-dimethylaminomethyl, N,N-diethylaminomethyl, 2-methoxyethoxymethyl, phenoxymethyl, 2-methoxyethoxy, 2-morpholin-4-ylethoxy, pyridin-3-ylmethoxy, 2-hydroxyethoxy, 2-N,N-dimethylaminoethoxy, methoxymethyl, 3-i-propoxymethyl, morpholin-4-ylmethyl, 3-hydroxypropyloxymethyl, 2-fluorophenoxymethyl, 3-fluorophenoxymethyl, 4-fluorophenoxy-methyl, 3-methoxypropyloxymethyl, pyridin-4-yloxymethyl, 2,4,6-trifluorophenoxymethyl, 2-oxopyridin-1-ylmethyl, 2,2,2-trifluoroethoxymethyl, 4-imidazol-1-ylphenoxymethyl, 4-[1.2.4-triazin-1-yl-phenoxymethyl, 2-phenylethyl, pyrrolidin-1-ylmethyl, piperidin-1-ylmethyl, 4-trifluoromethylpiperidin-1-ylmethyl, 4-methylpiperazin-1-ylmethyl, 3,3,3-trifluoropropyloxymethyl, 4-fluorophenylthiomethyl, 4-fluorophenylsulfinylmethyl, 4-fluorophenylsulfonylmethyl, pyridin-3-ylmethyloxymethyl, tetrahydropyran-4-yloxy, 2,2,2-trifluoroethyloxy, 2-pyrrolidin-1-ylethyloxy, piperidin-4-yloxy, N-methyl-N-benzylaminomethyl, N-methyl-N-2-phenylethylaminomethyl, 3-hydroxypropylthiomethyl, 3-hydroxypropylsulfinylmethyl, 3-hydroxypropylsulfonylmethyl, N-methyl-N-2-indol-3-ylethylaminomethyl, 2-(4-trifluoromethylphenyl)ethyl, 2-(3-trifluoromethoxyphenyl)ethyl, N-hydroxyaminocarbonyl-methylaminomethyl, or 3-(2-carboxyethylamino-methyl); or
a pharmaceutically acceptable salt thereof.

In some embodiments, Ar is benzofuran-2-yl and is monosubstituted at the 3-position of the benzofuran-2-yl ring with N,N-dimethylaminomethyl, N,N-diethylaminomethyl, 2-fluorophenoxymethyl, 3-fluorophenoxymethyl, 4-fluorophenoxy-methyl, hydroxyl-4-yloxymethyl, 2,4,6-trifluorophenoxy-methyl, 2-oxopyridin-1-ylmethyl, 2,2,2-trifluoroethoxymethyl, 4-imidazol-1-ylphenoxy-methyl, 4-[1.2.4-triazin-1-yl-phenoxymethyl, 2-phenylethyl, 3-hydroxypropyloxymethyl, 2-methoxyethyloxymethyl, pyrrolidin-1-ylmethyl, piperidin-1-ylmethyl, 4-trifluoromethylpiperidin-1-ylmethyl, 4-methylpiperazin-1-ylmethyl, 3,3,3-trifluoropropyloxymethyl, 4-fluorophenylthiomethyl, 4-fluorophenylsulfinylmethyl, 4-fluorophenylsulfonylmethyl, 2-(3-trifluoromethoxyphenylethyl), N-methyl-N-benzylaminomethyl, N-methyl-N-2-phenylethylaminomethyl, 3-hydroxypropyl-thiomethyl, 3-hydroxypropylsulfinyl-methyl, 3-hydroxypropylsulfonylmethyl, N-methyl-N-2-indol-3-ylethylaminomethyl, 2-(4-trifluoromethylphenyl)ethyl, N-hydroxyaminocarbonyl-methylaminomethyl, or 2-carboxyethylaminomethyl.

In some embodiments, Ar is benzofuran-2-yl and is monosubstituted at the 3-position of the benzofuran-2-yl ring with N,N-dimethylaminomethyl, N,N-diethylaminomethyl, 2-methoxyethoxymethyl, methoxymethyl, 3-i-propoxymethyl, morpholin-4-ylmethyl, 3-hydroxypropyloxymethyl, 3-methoxypropyloxymethyl, pyrrolidin-1-ylmethyl, or piperidin-1-ylmethyl.

In some embodiments, Ar is benzofuran-2-yl and is substituted at the 5-position of the benzofuran-2-yl ring with 1-cyclopropylpiperidin-4-yloxy, piperidin-4-yloxy, tetrahydropyran-4-yloxy, 2,2,2-trifluoroethoxy, 2-pyrrolidin-1-ylethyloxy, or 1-(2,2,2-trifluoroethyl)piperidin-4-yloxy.

In some embodiments, Ar is trans phenylCH═CH— wherein the phenyl is optionally substituted with one or two substituents independently selected from methyl, ethyl, methoxy, ethoxy, methylenedioxy, or —OH. In some embodiments, Ar is trans phenylCH═CH—.

In some embodiments, Ar is naphthyl wherein the naphthyl is optionally substituted with one or two substituents.

In some embodiments, Ar is quinolinyl wherein the quinolinyl is optionally substituted with one or two substituents.

In some embodiments, Ar is quinolinyl wherein the quinolinyl is optionally substituted with one or two substituents independently selected from chloro, fluoro, trifluoromethyl, methyl, ethyl, methoxy, ethoxy, methylenedioxy, —OH, 2-methoxyethoxy, 2-hydroxyethoxy, methoxymethyl, 3-i-propoxymethyl, 3-hydroxypropyloxymethyl, 3-methoxypropyloxymethyl, or 3,3,3-trifluoropropyloxymethyl.

In some embodiments, X is —O— and R³ is hydrogen.

In some embodiments, X is —S(O)ₙ and R³ is hydrogen.

In some embodiments, Y is ethylene. In some embodiments, Y is ethylene or —CH(C₂H₅)CH₂—. In some embodiments, Y is —CH(C₂H₅)CH₂—.

In some embodiments, X is —O—; R³ is hydrogen; and Y is ethylene or —CH(C₂H₅)CH₂—.

Yet other HDAC inhibitor compounds that are contemplated for use in the pharmaceutical compositions, pharmacokinetic strategies, dosing regimens, methods of treatments, and combination therapies include those compounds with the structure of Formula (II):

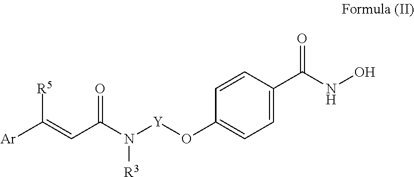

wherein:
X is —O—, —NR²—, or —S(O)ₙ where n is 0, 1, or 2 and R² is hydrogen, —CH₃, —CH₂CH₃;
Y is ethylene, propylene, 1-methylpropylene, 2-methylpropylene, —CH(C₂H₅)CH₂—, —CH(CH(CH₃)₂)CH₂—, and —CH(CH₃)CH₂—;
R³ is hydrogen, —CH₃, or —CH₂CH₃;
Ar is phenyl, naphthyl, quinolinyl, benzofuranyl, or benzothienyl, wherein Ar is optionally substituted with one or two substituents independently selected from chloro, fluoro, trifluoromethyl, methyl, ethyl, methoxy, ethoxy, methylenedioxy, —OH;
R⁵ is trifluoromethyl, methyl, ethyl, N,N-dimethylaminomethyl, N,N-diethylaminomethyl, 2-methoxyethoxymethyl, phenoxymethyl, methoxymethyl, 3-i-propoxymethyl, morpholin-4-ylmethyl, 3-hydroxypropyloxymethyl, 2-fluorophenoxymethyl, 3-fluorophenoxymethyl, 4-fluorophenoxy-methyl, 3-methoxypropyloxymethyl, pyridin-4-yloxymethyl, 2,4,6-trifluorophenoxymethyl, 2-oxopyridin-1-ylmethyl, 2,2,2-trifluoroethoxymethyl, 4-imidazol-1-ylphenoxymethyl, 2-phenylethyl, pyrrolidin-1-ylmethyl, piperidin-1-ylmethyl, 4-trifluoromethylpiperidin-1-ylmethyl, 4-methylpiperazin-1-ylmethyl, 3,3,3-trifluoropropyloxymethyl, 4-fluorophenylthiomethyl, 4-fluorophenylsulfinylmethyl, 4-fluorophenylsulfonylmethyl, pyridin-3-ylmethyloxymethyl, N-methyl-N-benzylaminomethyl, N-methyl-N-2-phenylethylaminomethyl, 3-hydroxypropylthiomethyl, 3-hydroxypropylsulfinylmethyl, 3-hydroxypropylsulfonylmethyl, N-methyl-N-2-indol-3-ylethylaminomethyl, 2-(4-trifluoromethylphenyl)ethyl, 2-(3-trifluoromethoxyphenyl)ethyl, N-hydroxyaminocarbonyl-methylaminomethyl, or 3-(2-carboxyethylamino-methyl); or a pharmaceutically acceptable salt thereof.

In some embodiments, Ar is benzofuranyl.

In some embodiments, R⁵ is N,N-dimethylaminomethyl, N,N-diethylaminomethyl, pyrrolidin-1-ylmethyl, or piperidin-1-ylmethyl.

In some embodiments, the HDAC inhibitor is selected from: N-hydroxy-4-[2-(4-methoxyquinolin-2-ylcarbonylamino)ethoxy]benzamide; N-hydroxy-4-[2S-(trans-cinnamoylamino)butoxy]benzamide; N-hydroxy-4-[2R-(trans-cinnamoylamino)butoxy]benzamide; N-hydroxy-4-{2-[4-(2-methoxyethoxyl)quinolin-2-ylcarbonylamino]ethoxy}benzamide; N-hydroxy-4-[2S-(benzothiophen-2-ylcarbonylamino)butoxy]-benzamide; N-hydroxy-4-{2S-[benzofuran-2-ylcarbonylamino]butoxy}benzamide; N-hydroxy-4-{2-[3-(methoxymethyl)benzofuran-2-ylcarbonylamino]ethoxy}benzamide; N-hydroxy-4-{2-[3-(N,N-dimethylaminomethyl)benzofuran-2-ylcarbonylamino]ethoxy}benzamide (abexinostat); N-hydroxy-4-{2-[3-(i-propoxymethyl)benzofuran-2-ylcarbonylamino]ethoxy}benzamide; N-hydroxy-4-{2-[3-(3-hydroxypropoxymethyl)benzofuran-2-ylcarbonylamino]ethoxy}-benzamide; N-hydroxy-4-{2-[3-(2-methoxyethyloxymethyl)benzofuran-2-ylcarbonylamino]ethoxy}-benzamide; N-hydroxy-4-{2-[3-(pyrrolidin-1-ylmethyl)benzofuran-2-ylcarbonylamino]ethoxy}-benzamide; N-hydroxy-4-{2-[3-(piperidin-1-ylmethyl)benzofuran-2-ylcarbonylamino]ethoxy}-benzamide; N-hydroxy-4-{2-[3-(4-methylpiperazin-1-ylmethyl)benzofuran-2-ylcarbonylamino]-ethoxy}benzamide; N-hydroxy-4-{2-[5-(tetrahydropyran-4-yloxy)benzofuran-2-ylcarbonylamino]ethoxy}-benzamide; N-hydroxy-4-{2-[5-(2-pyrrolidin-1-ylethyloxy)benzofuran-2-ylcarbonylamino]ethoxy}-benzamide; N-hydroxy-4-{2S-[5-(2-pyrrolidin-1-ylethyloxy)benzofuran-2-ylcarbonylamino]butoxy}-benzamide; N-hydroxy-4-{2-[5-(2-pyrrolidin-1-ylethyloxy)benzofuran-2-ylcarbonylamino]-1R-methyl-ethoxy}benzamide; and N-hydroxy-4-{2-[(3-(benzofuran-2-yl)-4-(dimethylamino)-but-2-enoyl)amino]-ethoxy}benzamide; or a pharmaceutically acceptable salt thereof.

In some embodiments, the HDAC inhibitor is N-hydroxy-4-{2-[3-(N,N-dimethylaminomethyl)benzofuran-2-ylcarbonylamino]ethoxy}-benzamide (abexinostat).

In some embodiments, the HDAC inhibitor is selected from HDAC inhibitors disclosed in WO 2004/092115 or WO 2005/097770, both of which are herein incorporated by reference.

### Forms and Phases

HDAC inhibitors (e.g. abexinostat), including pharmaceutically acceptable salts thereof, and pharmaceutically acceptable solvates thereof, are in various forms, including but not limited to, amorphous phase, partially crystalline forms, crystalline forms, milled forms, and nano-particulate forms. The crystalline forms are known as polymorphs. Polymorphs include the different crystal packing arrangements of the same elemental composition of a compound. This arrangement can significantly affect the physiochemical, formulation and processing parameters as well as the shelf life or stability of the substance and excipients. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature cause a single crystal form to dominate. In one aspect, a crystalline form of an HDAC inhibitor (e.g. abexinostat) is used in the pharmaceutical compositions disclosed herein. In one aspect, a crystalline form of the HCl salt of abexinostat is used in the pharmaceutical compositions disclosed herein. In one aspect, amorphous abexinostat is used in the pharmaceutical compositions disclosed herein. In one aspect, amorphous HCl salt of abexinostat is used in the pharmaceutical composition disclosed herein.

### Pharmaceutical Compositions

Disclosed herein, in certain embodiments, are methods of increasing the effectiveness of an antiangiogenic agent in an individual in need thereof, comprising co-administering to the individual (a) a cycle of abexinostat, or a salt thereof; and (b) an antiangiogenic agent. In some embodiments, the antiangiogenic agent is pazopanib or a salt thereof. In some embodiments, the method reduces resistance to the antiangiogenic agent; delays the development of resistance to the antiangiogenic agent; delays the onset of the cancer becoming refractory to the antiangiogenic agent; prolongs the usefulness of the antiangiogenic agent; allows use of the antiangiogenic agent in the treatment of cancers that generally develop, or have developed, resistance to the antiangiogenic agent; increases patient response to the antiangiogenic agent; increases cellular response to the antiangiogenic agent; decreases the effective dosage of the antiangiogenic agent; or any combination thereof.

Disclosed herein, in certain embodiments, are methods of increasing the effectiveness of pazopanib, or a salt thereof, in an individual in need thereof, comprising co-administering to the individual (a) a cycle of abexinostat, or a salt thereof; and (b) pazopanib, or a salt thereof. In some embodiments, the method reduces resistance to pazopanib, or a salt thereof; delays the development of resistance to pazopanib, or a salt thereof; delays the onset of the cancer becoming refractory to pazopanib, or a salt thereof; prolongs the usefulness of pazopanib, or a salt thereof; allows use of pazopanib, or a salt thereof, in the treatment of cancers that generally develop, or have developed, resistance to pazopanib, or a salt thereof; increases patient response to pazopanib, or a salt thereof; increases cellular response to pazopanib, or a salt thereof; decreases the effective dosage of pazopanib, or a salt thereof; or any combination thereof.

Additionally disclosed herein, in certain embodiments, are methods of treating cancer comprising administering (a) a cycle of abexinostate, or a salt thereof; and (b) an antiangiogenic agent. In some embodiments, the antiangiogenic agent is pazopanib, or a salt thereof. In some embodiments, the method reduces resistance to the antiangiogenic agent; delays the development of resistance to the antiangiogenic agent; delays the onset of the cancer becoming refractory to the antiangiogenic agent; prolongs the usefulness of the antiangiogenic agent; allows use of the antiangiogenic agent in the treatment of cancers that generally develop, or have developed, resistance to the antiangiogenic agent; increases patient response to the antiangiogenic agent; increases cellular response to the antiangiogenic agent; decreases the effective dosage of the antiangiogenic agent; or any combination thereof.

Further disclosed herein, in certain embodiments, are methods of treating cancer comprising administering (a) a cycle of abexinostate, or a salt thereof; and (b) pazopanib, or a salt thereof. In some embodiments, the method reduces resistance to pazopanib, or a salt thereof; delays the development of resistance to pazopanib, or a salt thereof; delays the onset of the cancer becoming refractory to pazopanib, or a salt thereof; prolongs the usefulness of pazopanib, or a salt thereof; allows use of pazopanib, or a salt thereof, in the treatment of cancers that generally develop, or have developed, resistance to pazopanib, or a salt thereof; increases patient response to pazopanib, or a salt thereof; increases cellular response to pazopanib, or a salt thereof; decreases the effective dosage of pazopanib, or a salt thereof; or any combination thereof.

Compositions for use with the methods disclosed herein are formulated in a conventional manner using one or more physiologically acceptable carriers (i.e. inactive ingredients) comprising excipients and auxiliaries which facilitate processing of the active compounds into preparations which are used pharmaceutically. Suitable techniques, carriers, and excipients include those found within, for example, Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference in their entirety.

Compositions for use with the methods disclosed herein comprise abexinostat (or a salt thereof), and/or pazopanib (or a salt thereof), and one or more of the following: (a) binders; (b) coatings; (c) disintegrants; (d) fillers (diluents); (e) lubricants; (f) glidants (flow enhancers); (g) compression aids; (h) colors; (i) sweeteners; (j) preservatives; (k) suspensing/dispersing agents; (l) film formers/coatings; (m) flavors; (n) printing inks; (o) gelling agents; (p) a second therapeutically active agent.

In one aspect, pharmaceutical compositions for use with the methods disclosed herein include one or more of the following in addition to the active agent(s) (e.g. abexinostat, a salt of abexinostat, pazopanib, and/or a salt of pazopanib): (a) magnesium stearate; (b) lactose; (c) microcrystalline Cellulose; (d) silicified microcrystalline cellulose; (e) mannitol; (f) starch (corn); (g) silicon dioxide; (h) titanium dioxide; (i) stearic acid; (j)s Starch glycolate; (k) gelatin; (l) talc; (m) sucrose; (n) aspartame; (o) calcium stearate; (p) povidone; (q) pregelatinized starch; (r) hydroxy propyl methylcellulose; (s) OPA products (coatings & inks); (t) croscarmellose; (u) hydroxy propyl cellulose; (v) ethylcellulose; (w) calcium phosphate (dibasic); (x) crospovidone; (y) shellac (and glaze); (z) sodium carbonate.

In some embodiments, pharmaceutical compositions for use with the methods disclosed herein comprise an active ingredient (e.g., abexinostat, a salt of abexinostat, pazopanib, and/or a salt of pazopanib) in a pharmaceutically acceptable vehicle, carrier, diluent, or excipient, or a mixture thereof; and one or more release controlling excipients as described herein. Suitable modified release dosage vehicles include, but are not limited to, hydrophilic or hydrophobic matrix devices, water-soluble separating layer coatings, enteric coatings, osmotic devices, multi-particulate devices, and combinations thereof. The pharmaceutical compositions may also comprise non-release controlling excipients.

In some embodiments, pharmaceutical compositions for use with the methods disclosed herein are film-coated dosage forms, which comprise a combination of an active ingredient and one or more tabletting excipients to form a tablet core using conventional tabletting processes and subsequently coating the core. The tablet cores can be produced using conventional granulation methods, for example wet or dry granulation, with optional comminution of the granules and with subsequent compression and coating. Granulation methods are described, for example, in Voigt, pages 156-69.

Suitable excipients for the production of granules are, for example pulverulent fillers optionally having flow-conditioning properties, for example talcum, silicon dioxide, for example synthetic amorphous anhydrous silicic acid of the Syloid® type (Grace), for example SYLOID 244 FP, microcrystalline cellulose, for example of the Avicel® type (FMC Corp.), for example of the types AVICEL PH101, 102, 105, RC581 or RC 591, Emcocel® type (Mendell Corp.) or Elcema® type (Degussa); carbohydrates, such as sugars, sugar alcohols, starches or starch derivatives, for example lactose, dextrose, saccharose, glucose, sorbitol, mannitol, xylitol, potato starch, maize starch, rice starch, wheat starch or amylopectin, tricalcium phosphate, calcium hydrogen phosphate or magnesium trisilicate; binders, such as gelatin, tragacanth, agar, alginic acid, cellulose ethers, for example methylcellulose, carboxymethylcellulose or hydroxypropylmethylcellulose, polyethylene glycols or ethylene oxide homopolymers, especially having a degree of polymerization of approximately from 2.0×10³ to 1.0×10⁵ and an approximate molecular weight of about from 1.0×10⁵ to 5.0×10⁶, for example excipients known by the name Polyox® (Union Carbide), polyvinylpyrrolidone or povidones, especially having a mean molecular weight of approximately 1000 and a degree of polymerization of approximately from about 500 to about 2500, and also agar or gelatin; surface-active substances, for example anionic surfactants of the alkyl sulfate type, for example sodium, potassium or magnesium n-dodecyl sulfate, n-tetradecyl sulfate, n-hexadecyl sulfate or n-octadecyl sulfate, of the alkyl ether sulfate type, for example sodium, potassium or magnesium n-dodecyloxyethyl sulfate, n-tetradecyloxyethyl sulfate, n-hexadecyloxyethyl sulfate or n-octadecyloxyethyl sulfate, or of the alkanesulfonate type, for example sodium, potassium or magnesium n-dodecanesulfonate, n-tetradecanesulfonate, n-hexadecanesulfonate or n-octadecanesulfonate, or non-ionic surfactants of the fatty acid polyhydroxy alcohol ester type, such as sorbitan monolaurate, monooleate, monostearate or monopalmitate, sorbitan tristearate or trioleate, polyoxyethylene adducts of fatty acid polyhydroxy alcohol esters, such as polyoxyethylene sorbitan monolaurate, monooleate, monostearate, monopalmitate, tristearate or trioleate, polyethylene glycol fatty acid esters, such as polyoxyethyl stearate, polyethylene glycol 400 stearate, polyethylene glycol 2000 stearate, especially ethylene oxide/propylene oxide block polymers of the Pluronics® (BWC) or Synperonic® (ICI) type

In some embodiments, pharmaceutical compositions for use with the methods disclosed herein are formulated in enteric coated dosage forms, which comprise a combination of an active ingredient, or a pharmaceutically acceptable salt, solvate, or prodrug thereof; and one or more release controlling excipients for use in an enteric coated dosage form. The pharmaceutical compositions may also comprise non-release controlling excipients.

In some embodiments, pharmaceutical compositions for use with the methods disclosed herein are formulated as a dosage form that has an instant releasing component and at least one delayed releasing component, and is capable of giving a discontinuous release of the compound in the form of at least two consecutive pulses separated in time from 0.5 hour up to 8 hours. The pharmaceutical compositions comprise a combination of an active ingredient, and one or more release controlling and non-release controlling excipients, such as those excipients suitable for a disruptable semi-permeable membrane and as swellable substances.

In some embodiments, pharmaceutical compositions for use with the methods disclosed herein are formulated as a dosage form for oral administration to a subject, which comprises a combination of an active ingredient; and one or more pharmaceutically acceptable excipients or carriers, enclosed in an intermediate reactive layer comprising a gastric juice-resistant polymeric layered material partially neutralized with alkali and having cation exchange capacity and a gastric juice-resistant outer layer.

In some embodiments, pharmaceutical compositions for use with the methods disclosed herein comprise an active ingredient, in the form of enteric-coated granules, as delayed-release capsules for oral administration.

The pharmaceutical compositions provided herein may be provided in unit-dosage forms or multiple-dosage forms. Unit-dosage forms, as used herein, refer to physically discrete units suitable for administration to human and animal subjects and packaged individually as is known in the art. Each unit-dose contains a predetermined quantity of the active ingredient(s) sufficient to produce the desired therapeutic effect, in association with the required pharmaceutical carriers or excipients. Examples of unit-dosage forms include individually packaged tablets and capsules. Unit-dosage forms may be administered in fractions or multiples thereof. A multiple-dosage form is a plurality of identical unit-dosage forms packaged in a single container to be administered in segregated unit-dosage form. Examples of multiple-dosage forms include bottles of tablets or capsules.

Pharmaceutical dosage forms can be formulated in a variety of methods and can provide a variety of drug release profiles, including immediate release, sustained release, and delayed release. In some cases it may be desirable to prevent drug release after drug administration until a certain amount of time has passed (i.e. timed release), to provide substantially continuous release over a predetermined time period (i.e. sustained release) or to provide release immediately following drug administration (i.e., immediate release).

Oral formulations are presented in the form of: tablets, capsules, pills, pellets, beads, granules, bulk powders. Capsules include mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. Tablet formulations are made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. In some embodiments are surface modifying agents which include nonionic and anionic surface modifying agents. For example, surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminum silicate, and triethanolamine.

In one aspect, oral formulations described herein utilize standard delay or time release formulations to alter the absorption of the active compound(s).

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL®-PH-101, AVICEL®-PH-103, AVICEL® RC-581, AVICEL®-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. Binder levels are from about 50% to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the pharmaceutical compositions provided herein varies upon the type of formulation, and is readily discernible to those of ordinary skill in the art. In one aspect, the pharmaceutical compositions provided herein include from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof. In one aspect, the pharmaceutical compositions provided herein include from about 0.1 to about 5% by weight of a lubricant.

Suitable glidants include colloidal silicon dioxide, CAB-O-SIL® (Cabot Co. of Boston, Mass.), and asbestos-free talc. Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye.

It should be understood that many carriers and excipients may serve several functions, even within the same formulation.

In some embodiments, pharmaceutical compositions for use with the methods disclosed herein are formulated as compressed tablets, tablet triturates, rapidly dissolving tablets, multiple compressed tablets, or enteric-coating tablets, sugar-coated, or film-coated tablets.

Enteric-coatings are coatings that resist the action of stomach acid but dissolve or disintegrate in the intestine.

In some embodiments, pharmaceutical compositions for use with the methods disclosed herein include an enteric coating(s). Enteric coatings include one or more of the following: cellulose acetate phthalate; methyl acrylate-methacrylic acid copolymers; cellulose acetate succinate; hydroxy propyl methyl cellulose phthalate; hydroxy propyl methyl cellulose acetate succinate (hypromellose acetate succinate); polyvinyl acetate phthalate (PVAP); methyl methacrylate-methacrylic acid copolymers; methacrylic acid copolymers, cellulose acetate (and its succinate and phthalate version); styrol maleic acid co-polymers; polymethacrylic acid/acrylic acid copolymer; hydroxyethyl ethyl cellulose phthalate; hydroxypropyl methyl cellulose acetate succinate; cellulose acetate tetrahydrophthalate; acrylic resin; shellac.

An enteric coating is a coating put on a tablet, pill, capsule, pellet, bead, granule, particle, etc. so that it doesn't dissolve until it reaches the small intestine.

Sugar-coated tablets are compressed tablets surrounded by a sugar coating, which may be beneficial in covering up objectionable tastes or odors and in protecting the tablets from oxidation.

Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms may be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

In some embodiments, pharmaceutical compositions for use with the methods disclosed herein are soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The capsules may also be coated as known by those of skill in the art in order to modify or sustain dissolution of the active ingredient.

Coloring and flavoring agents can be used in all of the above dosage forms.

In some embodiments, pharmaceutical compositions for use with the methods disclosed herein are formulated as immediate or modified release dosage forms, including delayed-, sustained, pulsed-, controlled, targeted-, and programmed-release forms.

In some embodiments, pharmaceutical compositions for use with the methods disclosed herein are in the form of immediate or modified release dosage forms, including delayed-, sustained, pulsed-, controlled, targeted-, and programmed-release forms.

### Controlled Release

In some embodiments, pharmaceutical compositions for use with the methods disclosed herein are in the form of a controlled release dosage form. As used herein, the term “controlled release” refers to a dosage form in which the rate or place of release of the active ingredient(s) is different from that of an immediate dosage form when orally administered. Controlled release dosage forms include delayed-, extended-, prolonged-, sustained-, pulsatile-, modified-, targeted-, programmed-release. The pharmaceutical compositions in controlled release dosage forms are prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, and combinations thereof. The release rate of the active ingredient(s) can also be modified by varying the particle sizes.

In some embodiments, pharmaceutical compositions for use with the methods disclosed herein are formulated to provide a controlled release of an active agent (e.g. abexinostat, a salt of abexinostat, pazopanib, and/or a salt of pazopanib), or a pharmaceutically acceptable salt thereof.

In contrast to immediate release compositions, controlled release compositions allow delivery of an agent to a human over an extended period of time according to a predetermined profile. Such release rates can provide therapeutically effective levels of agent for an extended period of time and thereby provide a longer period of pharmacologic response. Such longer periods of response provide for many inherent benefits that are not achieved with the corresponding short acting, immediate release preparations. In some embodiments, controlled release compositions provide therapeutically effective levels of the HDAC inhibitor (e.g. abexinostat) for an extended period of time and thereby provide a longer period of pharmacologic response.

In some embodiments, the solid dosage forms described herein can be formulated as enteric coated delayed release oral dosage forms, i.e., as an oral dosage form of a pharmaceutical composition as described herein which utilizes an enteric coating to affect release in the small intestine of the gastrointestinal tract. The enteric coated dosage form is a compressed or molded or extruded tablet/mold (coated or uncoated) containing granules, powder, pellets, beads or particles of the active ingredient and/or other composition components, which are themselves coated or uncoated. In one aspect, the enteric coated oral dosage form may is a capsule (coated or uncoated) containing pellets, beads or granules of the solid carrier or the composition, which are themselves coated or uncoated.

The term “delayed release” as used herein refers to the delivery so that the release can be accomplished at some generally predictable location in the intestinal tract more distal to that which would have been accomplished if there had been no delayed release alterations. In some embodiments the method for delay of release is coating. Any coatings should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the practice of the present invention to achieve delivery to the lower gastrointestinal tract. In some embodiments the polymers for use in the present invention are anionic carboxylic polymers. In other embodiments, the polymers and compatible mixtures thereof, and some of their properties, include, but are not limited to:

Shellac, also called purified lac. This coating dissolves in media of pH>7;

Acrylic polymers. The performance of acrylic polymers (primarily their solubility in biological fluids) can vary based on the degree and type of substitution. Examples of suitable acrylic polymers include methacrylic acid copolymers and ammonio methacrylate copolymers. The Eudragit series E, L, R, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine;

Cellulose Derivatives. Examples of suitable cellulose derivatives are: ethyl cellulose; reaction mixtures of partial acetate esters of cellulose with phthalic anhydride. The performance can vary based on the degree and type of substitution. Cellulose acetate phthalate (CAP) dissolves in pH>6. Aquateric (FMC) is an aqueous based system and is a spray dried CAP psuedolatex with particles <1 μm. Other components in Aquateric can include pluronics, Tweens, and acetylated monoglycerides. Other suitable cellulose derivatives include: cellulose acetate trimellitate (Eastman); methylcellulose (Pharmacoat, Methocel); hydroxypropylmethyl cellulose phthalate (HPMCP); hydroxypropylmethyl cellulose succinate (HPMCS); and hydroxypropylmethylcellulose acetate succinate (e.g., AQOAT (Shin Etsu)). The performance can vary based on the degree and type of substitution. For example, HPMCP such as, HP-50, HP-55, HP-55S, HP-55F grades are suitable. The performance can vary based on the degree and type of substitution. For example, suitable grades of hydroxypropylmethylcellulose acetate succinate include, but are not limited to, AS-LG (LF), which dissolves at pH 5, AS-MG (MF), which dissolves at pH 5.5, and AS-HG (HF), which dissolves at higher pH. These polymers are offered as granules, or as fine powders for aqueous dispersions;

Poly Vinyl Acetate Phthalate (PVAP). PVAP dissolves in pH>5, and it is much less permeable to water vapor and gastric fluids.

In some embodiments, the coating can, and usually does, contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate and triacetin. Conventional coating techniques such as spray or pan coating are employed to apply coatings. The coating thickness must be sufficient to ensure that the oral dosage form remains intact until the desired site of topical delivery in the intestinal tract is reached.

Colorants, detackifiers, surfactants, antifoaming agents, lubricants (e.g., carnuba wax or PEG) may be added to the coatings besides plasticizers to solubilize or disperse the coating material, and to improve coating performance and the coated product.

A particularly suitable methacrylic copolymer is Eudragit L®, particularly L-30D® and Eudragit 100-55®, manufactured by Rohm Pharma, Germany. In Eudragit L-30D®, the ratio of free carboxyl groups to ester groups is approximately 1:1. Further, the copolymer is known to be insoluble in gastrointestinal fluids having pH below 5.5, generally 1.5-5.5, i.e., the pH generally present in the fluid of the upper gastrointestinal tract, but readily soluble or partially soluble at pH above 5.5, i.e., the pH values present in the small intestine.

In some embodiments, materials include shellac, acrylic polymers, cellulosic derivatives, polyvinyl acetate phthalate, and mixtures thereof. In other embodiments, materials include Eudragit® series E, L, RL, RS, NE, L, L300, S, 100-55, cellulose acetate phthalate, Aquateric, cellulose acetate trimellitate, ethyl cellulose, hydroxypropyl methyl cellulose phthalate, hydroxypropyl methyl cellulose acetate succinate, poly vinyl acetate phthalate, and Cotteric.

For some types of drugs, it is preferred to release the drug in “pulses,” wherein a single dosage form provides for an initial dose of drug followed by a release-free interval, after which a second dose of drug is released, followed by one or more additional release-free intervals and drug release “pulses.” Alternatively, no drug is released for a period of time after administration of the dosage form, after which a dose of drug is released, followed by one or more additional release-free intervals and drug release “pulses.”

Pulsatile drug delivery is useful, for example, with active agents that have short half-lives are administered two or three times daily, with active agents that are extensively metabolized presystemically, and with active agents that should maintain certain plasma levels in order have optimized pharmacodynamic effects.

A pulsatile dosage form is capable of providing one or more immediate release pulses at predetermined time points after a controlled lag time or at specific sites. Pulsatile dosage forms including the formulations described herein, which include an HDAC inhibitor (e.g. abexinostat), or a pharmaceutically acceptable salt thereof, is administered using a variety of pulsatile formulations that have been described. For example, such formulations include, but are not limited to, those described in U.S. Pat. Nos. 5,011,692, 5,017,381, 5,229,135, 5,840,329, 4,871,549, 5,260,068, 5,260,069, 5,508,040, 5,567,441 and 5,837,284. In one embodiment, the controlled release dosage form is pulsatile release solid oral dosage form including at least two groups of particles, (i.e. multiparticulate) each containing the formulation described herein. The first group of particles provides a substantially immediate dose of an HDAC inhibitor (e.g. abexinostat), or a pharmaceutically acceptable salt thereof, upon ingestion by a mammal. The first group of particles can be either uncoated or include a coating and/or sealant. The second group of particles includes coated particles, which includes from about 2% to about 75%, preferably from about 2.5% to about 70%, and more preferably from about 40% to about 70%, by weight of the total dose of an HDAC inhibitor (e.g. abexinostat), or a pharmaceutically acceptable salt thereof, in said formulation, in admixture with one or more binders. The coating includes a pharmaceutically acceptable ingredient in an amount sufficient to provide a delay of from about 2 hours to about 7 hours following ingestion before release of the second dose. Suitable coatings include one or more differentially degradable coatings such as, by way of example only, pH sensitive coatings (enteric coatings) such as acrylic resins (e.g., Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, and Eudragit® NE30D, Eudragit® NE 40D) either alone or blended with cellulose derivatives, e.g., ethylcellulose, or non-enteric coatings having variable thickness to provide differential release of the formulation that includes an HDAC inhibitor (e.g. abexinostat), or a pharmaceutically acceptable salt thereof.

### Multiparticulate Controlled Release Devices

In some embodiments, pharmaceutical compositions for use with the methods disclosed herein are multiparticulate controlled release devices, which include a multiplicity of particles, granules, or pellets, ranging from about 10 μm to about 3 mm, about 50 μm to about 2.5 mm, or from about 100 μm to about 1 mm in diameter. Such multiparticulates are made by wet-granulation, dry-granulation, extrusion/spheronization, roller-compaction, melt-congealing, by spray-coating seed cores, and combinations thereof. See, for example, Multiparticulate Oral Drug Delivery; Marcel Dekker: 1994; and Pharmaceutical Pelletization Technology; Marcel Dekker: 1989.

Other excipients or carriers as described herein are blended with the pharmaceutical compositions to aid in processing and forming the multiparticulates. The resulting particles may themselves constitute the multiparticulate device or may be coated by various film-forming materials, such as enteric polymers, water-swellable, and water-soluble polymers. The multiparticulates can be further processed as a capsule or a tablet.

Intestinal protective drug absorption system (IPDAS) is a multiparticulate tablet technology that consists of high density controlled release beads that are compressed into a tablet form. The beads may be manufactured by techniques such as extrusion spheronization and controlled release can be achieved with the use of different polymer systems to coat the resultant beads. Alternatively, the drug can also be coated onto an inert carrier such as non-pareil seeds to produce instant release multiparticulates. Controlled release can be achieved by the formation of a polymeric membrane onto these instant release multiparticulates. Once an IPDAS tablet is ingested, it rapidly disintegrates and disperses beads containing the drug in the stomach which subsequently pass into the duodenum and along the gastrointestinal tract in a controlled and gradual manner, independent of the feeding state. Release of active ingredient from the multiparticulates occurs through a process of diffusion either through the polymeric membrane and/or the micro matrix of the polymer/active ingredient formed in the extruded/spheronized multiparticulates. The intestinal protection of IPDAS is by virtue of the multiparticulate nature of the formulation which ensures wide dispersion of drug throughout the gastrointestinal tract.

Spheroidal oral drug absorption system (SODAS) is a multiparticulate technology that enables the production of customized dosage forms and responds directly to individual drug candidate needs. It can provide a number of tailored drugs release profiles including immediate release of drug followed by sustained release to give rise to a fast onset of action which is maintained for at least 12 hours. Alternatively, the opposite scenario can be achieved where drug release is delayed for a number of hours.

Programmable oral drug absorption system (PRODAS) is presented as a number of mini tablets contained in hard gelatin capsule. It thus combines the benefits of tableting technology within a capsule. It is possible to incorporate many different minitablets, each one formulated individually and programmed to release drug at different sites within the gastrointestinal tract. These combinations may include immediate release, delayed release, and/or controlled release mini tablets. It is also possible to incorporate mini tablets of different sizes so that high drug loading is possible. Their size ranges usually from 1.5-4 mm in diameter.

Many other types of controlled release systems known to those of ordinary skill in the art and are suitable for use with the formulations described herein. Examples of such delivery systems include, e.g., polymer-based systems, such as polylactic and polyglycolic acid, polyanhydrides and polycaprolactone; porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, e.g., Liberman et al., Pharmaceutical Dosage Forms, 2 Ed., Vol. 1, pp. 209-214 (1990); Singh et al., Encyclopedia of Pharmaceutical Technology, 2^{nd} Ed., pp. 751-753 (2002); U.S. Pat. Nos. 4,327,725, 4,624,848, 4,968,509, 5,461,140, 5,456,923, 5,516,527, 5,622,721, 5,686,105, 5,700,410, 5,977,175, 6,465,014 and 6,932,983.

### Matrix Controlled Release Devices

In some embodiments, pharmaceutical compositions for use with the methods disclosed herein are in a modified release dosage form that is fabricated using a matrix controlled release device known to those skilled in the art (see, Takada et al in “Encyclopedia of Controlled Drug Delivery,” Vol. 2, Mathiowitz ed., Wiley, 1999).

In some embodiments, pharmaceutical compositions for use with the methods disclosed herein are formulated using an erodible matrix device, which is water-swellable, erodible, or soluble polymers, including synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; and cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

In some embodiments, pharmaceutical compositions for use with the methods disclosed herein are formulated with a non-erodible matrix device. The active ingredient(s) is dissolved or dispersed in an inert matrix and is released primarily by diffusion through the inert matrix once administered. Materials suitable for use as a non-erodible matrix device included, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinylacetate copolymers, ethylene/propylene copolymers, ethylene/ethyl acrylate copolymers, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, polyvinyl chloride, plasticized nylon, plasticized polyethyleneterephthalate, natural rubber, silicone rubbers, polydimethylsiloxanes, silicone carbonate copolymers, and; hydrophilic polymers, such as ethyl cellulose, cellulose acetate, crospovidone, and cross-linked partially hydrolyzed polyvinyl acetate, and fatty compounds, such as carnauba wax, microcrystalline wax, and triglycerides.

In a matrix controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients or carriers in the compositions.

In one aspect, modified release dosage forms are prepared by methods known to those skilled in the art, including direct compression, dry or wet granulation followed by compression, melt-granulation followed by compression.

In some embodiments, a matrix controlled release system includes an enteric coating so that no drug is released in the stomach.

### Osmotic Controlled Release Devices

In some embodiments, pharmaceutical compositions for use with the methods disclosed herein are fabricated using an osmotic controlled release device, including one-chamber system, two-chamber system, asymmetric membrane technology (AMT), and extruding core system (ECS). In general, such devices have at least two components: (a) the core which contains the active ingredient(s); and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents water-swellable hydrophilic polymers, which are also referred to as “osmopolymers” and “hydrogels,” including, but not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents are osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol, organic acids, such as ascorbic acid, benzoic acid, fumaric acid, citric acid, maleic acid, sebacic acid, sorbic acid, adipic acid, edetic acid, glutamic acid, p-tolunesulfonic acid, succinic acid, and tartaric acid; urea; and mixtures thereof.

Osmotic agents of different dissolution rates may be employed to influence how rapidly the active ingredient(s) is initially delivered from the dosage form. For example, amorphous sugars, such as Mannogeme EZ (SPI Pharma, Lewes, Del.) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core may also include a wide variety of other excipients and carriers as described herein to enhance the performance of the dosage form or to promote stability or processing.

Materials useful in forming the semi-permeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semi-permeable membrane may also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semi-permeable membrane may be formed post-coating by mechanical or laser drilling. Delivery port(s) may also be formed in situ by erosion of a plug of water-soluble material or by rupture of a thinner portion of the membrane over an indentation in the core. In addition, delivery ports may be formed during coating process, as in the case of asymmetric membrane coatings of the type disclosed in U.S. Pat. Nos. 5,612,059 and 5,698,220.

The total amount of the active ingredient(s) released and the release rate can substantially by modulated via the thickness and porosity of the semi-permeable membrane, the composition of the core, and the number, size, and position of the delivery ports.

The pharmaceutical compositions in an osmotic controlled-release dosage form may further comprise additional conventional excipients or carriers as described herein to promote performance or processing of the formulation.

The osmotic controlled-release dosage forms can be prepared according to conventional methods and techniques known to those skilled in the art (see, Remington: The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27).

In other embodiments, pharmaceutical compositions provided herein are formulated as AMT controlled-release dosage form, which comprises an asymmetric osmotic membrane that coats a core comprising the active ingredient(s) and other pharmaceutically acceptable excipients or carriers. See U.S. Pat. No. 5,612,059 and WO 2002/17918. The AMT controlled-release dosage forms can be prepared according to conventional methods and techniques known to those skilled in the art, including direct compression, dry granulation, wet granulation, and a dip-coating method.

In certain embodiments, the pharmaceutical compositions provided herein are formulated as ESC controlled-release dosage form, which comprises an osmotic membrane that coats a core comprising the active ingredient(s), a hydroxylethyl cellulose, and other pharmaceutically acceptable excipients or carriers.

### Multilayered Tablets

In some embodiments, pharmaceutical compositions for use with the methods disclosed herein are in the form of a multilayered tablet. Multilayered tablets include an inert core, onto which is applied a layered of drug (plus optional excipients), followed by an enteric coating. A second layer of drug is applied onto the first enteric coating followed by a second enteric coating on the second layer of drug. The enteric coatings should ensure that the release of drug from each layer is separated in time by at least 3-6 hours.

### Immediate Release

In some embodiments, pharmaceutical compositions for use with the methods disclosed herein are immediate release dosage form capable of releasing not less than 75% of the therapeutically active ingredient or combination and/or meet the disintegration or dissolution requirements for immediate release tablets of the particular therapeutic agents or combination included in the tablet core, as set forth in USP XXII, 1990 (The United States Pharmacopeia). Immediate release pharmaceutical compositions include capsules, tablets, oral solutions, powders, beads, pellets, particles, and the like.

### Parenteral Administration

In some embodiments, pharmaceutical compositions for use with the methods disclosed herein are administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, and subcutaneous administration.

In some embodiments, pharmaceutical compositions for use with the methods disclosed herein are formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration may include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, dimethylacetamide, and dimethylsulfoxide.

Suitable antimicrobial agents or preservatives include, but are not limited to, phenols, cresols, mercurials, benzyl alcohol, chlorobutanol, methyl and propyl p-hydroxybenzates, thimerosal, benzalkonium chloride, benzethonium chloride, methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including a-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

In some embodiments, pharmaceutical compositions for use with the methods disclosed herein are formulated for single or multiple dosage administration. The single dosage formulations are packaged in an ampule, a vial, or a syringe. The multiple dosage parenteral formulations must contain an antimicrobial agent at bacteriostatic or fungistatic concentrations. All parenteral formulations must be sterile, as known and practiced in the art.

In some embodiments, pharmaceutical compositions for use with the methods disclosed herein are provided as ready-to-use sterile solutions. In some embodiments, pharmaceutical compositions for use with the methods disclosed herein are provided as sterile dry soluble products, including lyophilized powders and hypodermic tablets, to be reconstituted with a vehicle prior to use. In yet another embodiment, pharmaceutical compositions for use with the methods disclosed herein are provided as ready-to-use sterile suspensions. In yet another embodiment, pharmaceutical compositions for use with the methods disclosed herein are provided as sterile dry insoluble products to be reconstituted with a vehicle prior to use. In still another embodiment, pharmaceutical compositions for use with the methods disclosed herein are provided as ready-to-use sterile emulsions.

### Cancers

Disclosed herein, in certain embodiments, are methods of increasing the effectiveness of an antiangiogenic agent in an individual in need thereof, comprising co-administering to the individual (a) a cycle of abexinostat, or a salt thereof; and (b) an antiangiogenic agent. In some embodiments, the antiangiogenic agent is pazopanib or a salt thereof. In some embodiments, the method reduces resistance to the antiangiogenic agent; delays the development of resistance to the antiangiogenic agent; delays the onset of the cancer becoming refractory to the antiangiogenic agent; prolongs the usefulness of the antiangiogenic agent; allows use of the antiangiogenic agent in the treatment of cancers that generally develop, or have developed, resistance to the antiangiogenic agent; increases patient response to the antiangiogenic agent; increases cellular response to the antiangiogenic agent; decreases the effective dosage of the antiangiogenic agent; or any combination thereof.

Disclosed herein, in certain embodiments, are methods of increasing the effectiveness of pazopanib, or a salt thereof, in an individual in need thereof, comprising co-administering to the individual (a) a cycle of abexinostat, or a salt thereof; and (b) pazopanib, or a salt thereof. In some embodiments, the method reduces resistance to pazopanib, or a salt thereof; delays the development of resistance to pazopanib, or a salt thereof; delays the onset of the cancer becoming refractory to pazopanib, or a salt thereof; prolongs the usefulness of pazopanib, or a salt thereof; allows use of pazopanib, or a salt thereof, in the treatment of cancers that generally develop, or have developed, resistance to pazopanib, or a salt thereof; increases patient response to pazopanib, or a salt thereof; increases cellular response to pazopanib, or a salt thereof; decreases the effective dosage of pazopanib, or a salt thereof; or any combination thereof.

Additionally disclosed herein, in certain embodiments, are methods of treating cancer comprising administering (a) a cycle of abexinostate, or a salt thereof; and (b) an antiangiogenic agent. In some embodiments, the antiangiogenic agent is pazopanib, or a salt thereof. In some embodiments, the method reduces resistance to the antiangiogenic agent; delays the development of resistance to the antiangiogenic agent; delays the onset of the cancer becoming refractory to the antiangiogenic agent; prolongs the usefulness of the antiangiogenic agent; allows use of the antiangiogenic agent in the treatment of cancers that generally develop, or have developed, resistance to the antiangiogenic agent; increases patient response to the antiangiogenic agent; increases cellular response to the antiangiogenic agent; decreases the effective dosage of the antiangiogenic agent; or any combination thereof.

Further disclosed herein, in certain embodiments, are methods of treating cancer comprising administering (a) a cycle of abexinostate, or a salt thereof; and (b) pazopanib, or a salt thereof. In some embodiments, the method reduces resistance to pazopanib, or a salt thereof; delays the development of resistance to pazopanib, or a salt thereof; delays the onset of the cancer becoming refractory to pazopanib, or a salt thereof; prolongs the usefulness of pazopanib, or a salt thereof; allows use of pazopanib, or a salt thereof, in the treatment of cancers that generally develop, or have developed, resistance to pazopanib, or a salt thereof; increases patient response to pazopanib, or a salt thereof; increases cellular response to pazopanib, or a salt thereof; decreases the effective dosage of pazopanib, or a salt thereof; or any combination thereof.

In some embodiments, the methods disclosed herein are used in the treatment of cancer in a human. In some embodiments, the methods disclosed herein are used in the treatment of a hematological cancer in a human. In some embodiments, the methods disclosed herein are used in the treatment of a solid tumor in a human.

Hematological cancers include cancers of the blood or bone marrow, such as leukemia or lymphoma.

A lymphoma is a cancer that begins in cells of the immune system. There are two basic categories of lymphomas. One kind is Hodgkin lymphoma, which is marked by the presence of a type of cell called the Reed-Sternberg cell. The other category is non-Hodgkin lymphomas, which includes a large, diverse group of cancers of immune system cells. Non-Hodgkin lymphomas can be further divided into cancers that have an indolent (slow-growing) course and those that have an aggressive (fast-growing) course.

A leukemia is a cancer that starts in blood-forming tissue such as the bone marrow and causes large numbers of blood cells to be produced and enter the bloodstream.

In one aspect, the cancer is a solid tumor or a lymphoma or leukemia. In one aspect, the cancer is a carcinoma, a sarcoma, a lymphoma, a leukemia, a germ cell tumor, a blastic tumor or blastoma.

In some embodiments, the methods disclosed herein are used in the treatment of a solid tumor. In some embodiments, the methods disclosed herein are used in the treatment of a metstatic solid tumor. In some embodiments, the methods disclosed herein are used in the treatment of an advanced solid tumor.

In some embodiments, the methods disclosed herein are used in the treatment of a sarcoma.

In some embodiments, the methods disclosed herein are used in the treatment of a cancer selected from: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastom, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor, chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiforme, oligodendroglioma, schwannoma, retinoblastoma, congenital tumors), spinal cord (neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, endometrioid tumors, celioblastoma, clear cell carcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma [embryonal rhabdomyosarcoma], fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles, dysplastic nevi, lipoma, angioma, dermatofibroma, keloids, psoriasis; Adrenal glands: neuroblastoma; gallbladder carcinomas.

In one aspect, the cancer is breast cancer, colon cancer, colorectal carcinomas, non-small cell lung cancer, small-cell lung cancer, liver cancer, ovarian cancer, prostate cancer, uterine cervix cancer, urinary bladder cancer, gastric carcinomas, gastrointestinal stromal tumors, pancreatic cancer, germ cell tumors, mast cell tumors, neuroblastoma, mastocytosis, testicular cancers, glioblastomas, astrocytomas, lymphomas, melanoma, myelomas, acute myelocytic leukemia (AML), acute lymphocytic leukemia (ALL), myelodysplastic syndrome, and chronic myelogenous leukemia (CML).

In some embodiments, the cancer is a renal cell carcinoma.

In some embodiments, the cancer is ovarian cancer.

In one aspect, the cancer is a lymphoma. In one aspect, the lymphoma is a B cell lymphoma, T cell lymphoma, Hodgkin's lymphoma, or non-Hodgkin's lymphoma.

In one aspect, the cancer is a T-cell lymphoma or leukemia.

In one aspect, the T-cell lymphoma is peripheral T cell lymphoma. In another aspect, the T-cell lymphoma or leukemia is T cell lymphoblastic leukemia/lymphoma. In yet another aspect, the T-cell lymphoma is cutaneous T cell lymphoma. In another aspect, the T-cell lymphoma is adult T cell lymphoma. In one aspect, the T-cell lymphoma is peripheral T cell lymphoma, lymphoblastic lymphoma, cutaneous T cell lymphoma, NK/T-cell lymphoma, or adult T cell leukemia/lymphoma.

In one embodiment, the cancer is a sarcoma. A sarcoma is a cancer that begins in the muscle, fat, fibrous tissue, blood vessels, or other supporting tissue of the body. Sarcomas include any one of the following: alveolar soft part sarcoma, angiosarcoma, dermatofibrosarcoma, desmoid tumor, desmoplastic small round cell tumor, extraskeletal chondrosarcoma, extraskeletal osteosarcoma, fibrosarcoma, hemangiopericytoma, hemangiosarcoma, kaposi's sarcoma, leiomyosarcoma, liposarcoma, lymphangiosarcoma, malignant fibrous histiocytoma, neurofibrosarcoma, rhabdomyosarcoma, synovial sarcoma, askin's tumor, ewing's, malignant hemangioendothelioma, malignant schwannoma, osteosarcoma, chondrosarcoma. In some embodiments, the sarcoma is a soft-tissue sarcoma.

In some embodiments, the methods disclosed herein are used in the treatment of a soft tissue sarcoma in a human.

In some embodiments, the methods disclosed herein are used in the treatment of myelodysplastic syndrome (MDS) in a human.

In some embodiments, the methods disclosed herein are used in the treatment of chronic myelogenous leukemia (CML) in a human.

In some embodiments, the methods disclosed herein are used in the treatment of non-Hodgkin lymphoma in a human. In some embodiments, the methods disclosed herein are used in the treatment of Hodgkin Disease in a human.

In some embodiments, the methods disclosed herein are used in the treatment of multiple myeloma in a human.

In some embodiments, the methods disclosed herein are used in the treatment of chronic lymphocytic leukemia. In some embodiments, the methods disclosed herein are used in the treatment of acute lymphocytic leukemia.

In some embodiments, the methods disclosed herein are used in the treatment of a solid tumor in a human.

In some embodiments, the methods disclosed herein are used in the treatment of a sarcoma in a human.

### Combination Therapies

Disclosed herein, in certain embodiments, are methods of increasing the effectiveness of an antiangiogenic agent in an individual in need thereof, comprising co-administering to the individual (a) a cycle of abexinostat, or a salt thereof; and (b) an antiangiogenic agent. In some embodiments, the antiangiogenic agent is pazopanib or a salt thereof. In some embodiments, the method reduces resistance to the antiangiogenic agent; delays the development of resistance to the antiangiogenic agent; delays the onset of the cancer becoming refractory to the antiangiogenic agent; prolongs the usefulness of the antiangiogenic agent; allows use of the antiangiogenic agent in the treatment of cancers that generally develop, or have developed, resistance to the antiangiogenic agent; increases patient response to the antiangiogenic agent; increases cellular response to the antiangiogenic agent; decreases the effective dosage of the antiangiogenic agent; or any combination thereof.

Disclosed herein, in certain embodiments, are methods of increasing the effectiveness of pazopanib, or a salt thereof, in an individual in need thereof, comprising co-administering to the individual (a) a cycle of abexinostat, or a salt thereof; and (b) pazopanib, or a salt thereof. In some embodiments, the method reduces resistance to pazopanib, or a salt thereof; delays the development of resistance to pazopanib, or a salt thereof; delays the onset of the cancer becoming refractory to pazopanib, or a salt thereof; prolongs the usefulness of pazopanib, or a salt thereof; allows use of pazopanib, or a salt thereof, in the treatment of cancers that generally develop, or have developed, resistance to pazopanib, or a salt thereof; increases patient response to pazopanib, or a salt thereof; increases cellular response to pazopanib, or a salt thereof; decreases the effective dosage of pazopanib, or a salt thereof; or any combination thereof.

Additionally disclosed herein, in certain embodiments, are methods of treating cancer comprising administering (a) a cycle of abexinostate, or a salt thereof; and (b) an antiangiogenic agent. In some embodiments, the antiangiogenic agent is pazopanib, or a salt thereof. In some embodiments, the method reduces resistance to the antiangiogenic agent; delays the development of resistance to the antiangiogenic agent; delays the onset of the cancer becoming refractory to the antiangiogenic agent; prolongs the usefulness of the antiangiogenic agent; allows use of the antiangiogenic agent in the treatment of cancers that generally develop, or have developed, resistance to the antiangiogenic agent; increases patient response to the antiangiogenic agent; increases cellular response to the antiangiogenic agent; decreases the effective dosage of the antiangiogenic agent; or any combination thereof.

Further disclosed herein, in certain embodiments, are methods of treating cancer comprising administering (a) a cycle of abexinostate, or a salt thereof; and (b) pazopanib, or a salt thereof. In some embodiments, the method reduces resistance to pazopanib, or a salt thereof; delays the development of resistance to pazopanib, or a salt thereof; delays the onset of the cancer becoming refractory to pazopanib, or a salt thereof; prolongs the usefulness of pazopanib, or a salt thereof; allows use of pazopanib, or a salt thereof, in the treatment of cancers that generally develop, or have developed, resistance to pazopanib, or a salt thereof; increases patient response to pazopanib, or a salt thereof; increases cellular response to pazopanib, or a salt thereof; decreases the effective dosage of pazopanib, or a salt thereof; or any combination thereof.

In one embodiment, the compositions and methods described herein are also used in conjunction with other therapeutic reagents that are selected for their particular usefulness against the cancer that is being treated. In general, the compositions described herein and, in embodiments where combinational therapy is employed, other agents do not have to be administered in the same pharmaceutical composition, and are, because of different physical and chemical characteristics, administered by different routes. In one embodiment, the initial administration is made according to established protocols, and then, based upon the observed effects, the dosage, modes of administration and times of administration, further modified.

In certain embodiments, the particular choice of compounds used depends on the diagnosis of the attending physicians and their judgment of the condition of the patient and the appropriate treatment protocol. In various embodiments, the compounds are administered concurrently (e.g., simultaneously, essentially simultaneously or within the same treatment protocol) or sequentially, depending upon the nature of the cancer, the condition of the patient, and the actual choice of compounds used. In certain embodiments, the determination of the order of administration, and the number of repetitions of administration of each therapeutic agent during a treatment protocol, is based upon evaluation of the disease being treated and the condition of the patient.

In one embodiment, it is understood that the dosage regimen to treat the cancer is modified in accordance with a variety of factors. These factors include the type of cancer from which the human suffers, as well as the age, weight, sex, diet, and medical condition of the human. Thus, in one embodiment, the dosage regimen actually employed varies widely and therefore deviates from the dosage regimens set forth herein. In certain embodiments, treatment of a cancer with a combination of an HDAC inhibitor (e.g. abexinostat) and a second agent allows for the effective amount of the HDAC inhibitor (e.g. abexinostat) and/or the second agent to be decreased.

The formulations described herein are administered and dosed in accordance with good medical practice, taking into account the clinical condition of the individual patient, the method of administration, scheduling of administration, and other factors known to medical practitioners.

Contemplated pharmaceutical compositions provide a therapeutically effective amount of an HDAC inhibitor (e.g. abexinostat) enabling, for example, once-a-day, twice-a-day, three times a day, etc. administration. In one aspect, pharmaceutical compositions provide an effective amount of an HDAC inhibitor (e.g. abexinostat) enabling once-a-day dosing.

In some embodiments, the methods disclosed herein further comprise administering an additional agent in combination with abexinostat (or a salt thereof), and pazopanib (or a salt thereof).

In certain embodiments, the therapeutic effectiveness of the methods disclosed herein is enhanced by administration of an adjuvant (i.e., by itself the adjuvant has minimal therapeutic benefit, but in combination with another therapeutic agent, the overall therapeutic benefit to the patient is enhanced). In some embodiments, the benefit experienced by a patient is increased by administering an another therapeutic agent (which also includes a therapeutic regimen) that also has therapeutic benefit. In specific embodiments, increased therapeutic benefit results by also providing the patient with other therapeutic agents or therapies for cancer. In various embodiments, use of an additional agent provides the individual with, e.g., an additive or synergistic benefit.

Therapeutically-effective dosages vary when the drugs are used in treatment combinations. Determination of therapeutically-effective dosages of drugs and other agents when used in combination treatment regimens is achieved in any manner. For example, the use of metronomic dosing, i.e., providing more frequent, lower doses in order to minimize toxic side effects can be utilized. In certain instances, the combination therapy allows for any or all of the active agents to have a therapeutically effective amount that is lower than would be obtained when administering either agent alone.

A combination treatment regimen encompasses, by way of non-limiting example, treatment regimens in which administration of abexinostat (or a salt thereof), and pazopanib (or a salt thereof) is initiated prior to, during, or after treatment with an additional agent, and continues until any time during treatment with the additional agent or after termination of treatment with the additional agent. It also includes treatments in which abexinostat (or a salt thereof), and pazopanib (or a salt thereof) and the additional agent being used in combination are administered simultaneously or at different times and/or at decreasing or increasing intervals during the treatment period. Combination treatment further includes periodic treatments that start and stop at various times to assist with the clinical management of the patient.

In any case, the multiple therapeutic agents are administered in any order, including, e.g., simultaneously. If administration is simultaneous, the multiple therapeutic agents are provided, in various embodiments, in a single, unified form, or in multiple forms (by way of example only, either as a single pill or as two separate pills). In various embodiments, one of the therapeutic agents is given in multiple doses, or both are given as multiple doses. In certain embodiments wherein administration of the multiple agents is not simultaneous, the timing between administration of the multiple agents is of any acceptable range including, e.g., from more than zero weeks to less than four weeks. Any number of additional agents may be used in combination with the methods disclosed herein,

In certain embodiments, the initial administration is via oral administration, such as, for example, a pill, a capsule, a tablet, a solution, a suspension, and the like, or combination thereof. In certain embodiments, the methods disclosed herein are used as soon as is practicable after the onset of a cancer is detected or suspected, and for a length of time necessary for the treatment of the cancer. In certain embodiments, the methods disclosed herein are continued for any length of time necessary for the treatment of the cancer including, by way of non limiting example, for at least 2 weeks, at least 1 month, or more than 1 month.

Additional therapeutic agents are selected from among DNA-damaging agents; topoisomerase I or II inhibitors; alkylating agents; PARP inhibitors; proteasome inhibitors; RNA/DNA antimetabolites; antimitotics; immunomodulatory agents; antiangiogenics; aromatase inhibitors; hormone-modulating agents; apoptosis inducing agents; kinase inhibitors; monoclonal antibodies; abarelix; ABT-888; aldesleukin; aldesleukin; alemtuzumab; alitretinoin; allopurinol; altretamine; amifostine anastrozole; arsenic trioxide; asparaginase; azacitidine; AZD-2281; bendamustine; bevacizumab; bexarotene; bleomycin; bortezomib; BSI-201; busulfan; busulfan; calusterone; capecitabine; carboplatin; carfilozib; carmustine; carmustine; celecoxib; cetuximab; chlorambucil; cisplatin; cladribine; clofarabine; cyclophosphamide; cytarabine; cytarabine liposomal; dacarbazine; dactinomycin; darbepoetin alfa; dasatinib; daunorubicin liposomal; daunorubicin; decitabine; denileukin; dexrazoxane; docetaxel; doxorubicin; doxorubicin liposomal; dromostanolone propionate; epirubicin; epoetin alfa; erlotinib; estramustine; etoposide phosphate; etoposide; exemestane; filgrastim; floxuridine; fludarabine; fluorouracil; fulvestrant; gefitinib; gemcitabine; gemtuzumab ozogamicin; goserelin acetate; histrelin acetate; hydroxyurea; Ibritumomab tiuxetan; idarubicin; ifosfamide; imatinib mesylate; interferon alfa 2a; Interferon alfa-2b; irinotecan; lenalidomide; letrozole; leucovorin; leuprolide Acetate; levamisole; lomustine; meclorethamine; megestrol acetate; melphalan; mercaptopurine; methotrexate; methoxsalen; mitomycin C; mitomycin C; mitotane; mitoxantrone; nandrolone phenpropionate; nelarabine; NPI-0052; nofetumomab; oprelvekin; oxaliplatin; paclitaxel; paclitaxel protein-bound particles; palifermin; pamidronate; panitumumab; pegademase; pegaspargase; pegfilgrastim; pemetrexed disodium; pentostatin; pipobroman; plicamycin, mithramycin; porfimer sodium; procarbazine; quinacrine; RAD001; rasburicase; rituximab; sargramostim; Sargramostim; sorafenib; streptozocin; sunitinib malate; tamoxifen; temozolomide; teniposide; testolactone; thalidomide; thioguanine; thiotepa; topotecan; toremifene; tositumomab; tositumomab/I-131 tositumomab; trastuzumab; tretinoin; uracil Mustard; valrubicin; vinblastine; vincristine; vinorelbine; vorinostat; zoledronate; and zoledronic acid.

In some embodiments, the additional agent is a topoisomerase inhibitor, tubulin interactor, DNA-interactive agent, DNA-alkylating agent, and/or platinum complex.

In some embodiments, the additional agent is oxaliplatin, tyrosine kinase inhibitor, irinotecan (CPT-11), azacitidine, fludaribine, or bendamustine.

Tyrosine kinase inhibitors include, but are not limited to, erlotinib, gefitinib, lapatinib, vandetanib, neratinib, lapatinib, neratinib, axitinib, sunitinib, sorafenib, lestaurtinib, semaxanib, cediranib, imatinib, nilotinib, dasatinib, bosutinib, lestaurtinib, vatalanib and soratinib.

In some embodiments, the additional agent is a DNA damaging anti-cancer agent and/or radiation therapy.

DNA damaging anti-cancer agents and/or radiation therapy include, but is not limited to, ionizing radiation, radiomimetic drugs, monofunctional alkylators (e.g. alkylsulphonates, nitrosoureas, temozolomide), bifunctional alkylators (nitrogen mustard, mitomycin C, cisplatin), antimetabolites (e.g. 5-fluorouracil, thiopurines, folate analogues), topoisomerase inhibitors (e.g. camptothecins, etoposide, doxorubicin), replication inhibitors (e.g. aphidicolin, hydroxyurea), cytotoxic/cytostatic agents, antiproliferative agents, prenyl-protein transferase inhibitors, nitrogen mustards, nitroso ureas, angiogenesis inhibitors, inhibitors of cell proliferation and survival signaling pathway, apoptosis inducing agents, agents that interfere with cell cycle checkpoints, biphosphonates, or any combination thereof.

In some embodiments, the additional agent is an inhibitor of inherent multidrug resistance (MDR), in particular MDR associated with high levels of expression of transporter proteins. Such MDR inhibitors include inhibitors of p-glycoprotein (P-gp), such as LY335979, XR9576, OC144-093, R101922, VX853 and PSC833 (valspodar).

In some embodiments, the additional agent is anti-emetic agents to treat nausea or emesis, including acute, delayed, late-phase, and anticipatory emesis, which may result from the use of an HDAC inhibitor (e.g. abexinostat), alone or with radiation therapy. Anti-emetic agents include neurokinin-1 receptor antagonists, 5HT3 receptor antagonists (such as ondansetron, granisetron, tropisetron, Palonosetron, and zatisetron), GABA_{B} receptor agonists (such as baclofen), corticosteroids (such as dexamethasone, prednisone, prednisolone, or others such as disclosed in U.S. Pat. Nos. 2,789,118; 2,990,401; 3,048,581; 3,126,375; 3,929,768; 3,996,359; 3,928,326 and 3,749,712), dopamine antagonists (such as, domperidone, droperidol, haloperidol, chlorpromazine, promethazine, prochlorperazine, metoclopramide), antihistamines (H1 histamine receptor antagonists, such as cyclizine, diphenhydramine, dimenhydrinate, meclizine, promethazine, hydroxyzine), cannabinoids (such as cannabis, marinol, dronabinol), and others (such as trimethobenzamide; ginger, emetrol, propofol).

In some embodiments, the additional agent is an anti-emesis agent selected from among a neurokinin-1 receptor antagonist, a 5HT3 receptor antagonist and a corticosteroid.

In some embodiments, the additional agent is an agent useful in the treatment of anemia. Such an anemia treatment agent is, for example, a continuous eythropoiesis receptor activator (such as epoetin-α).

In some embodiments, the additional agent is an agent useful in the treatment of neutropenia. Examples of agents useful in the treatment of neutropenia include, but are not limited to, a hematopoietic growth factor which regulates the production and function of neutrophils such as a human granulocyte colony stimulating factor, (G-CSF). Examples of a G-CSF include filgrastim.

In some embodiments, the additional agent is an inhibitor of at least one CYP enzyme. In situations where the abexinostat (or a salt thereof), or pazopanib (or a salt thereof) is metabolized by one or more CYP enzymes, coadministration with a CYP inhibitor reduces in vivo metabolism and improves the pharmacokinetic properties of the agent.

Other combination therapies are disclosed in WO 08/082856 and WO 07/109178, both of which are herein incorporated by reference in their entirety.

### Radiation Therapy

In some embodiments, the methods disclosed herein further comprise radiation therapy. Radiation therapy, also called radiotherapy, is the treatment of cancer and other diseases with ionizing radiation. Ionizing radiation deposits energy that injures or destroys cells in an area being treated (a “target tissue”) by damaging their genetic material, making it impossible for these cells to continue to grow. Although radiation damages both cancer cells and normal cells, the latter are better able to repair themselves and function properly. Radiotherapy can be used to treat localized solid tumors, such as cancers of the skin, tongue, larynx, brain, breast, prostate, colon, uterus and/or cervix. It can also be used to treat leukemia and lymphoma (cancers of the blood-forming cells and lymphatic system, respectively).

A technique for delivering radiation to cancer cells is to place radioactive implants directly in a tumor or body cavity. This is called internal radiotherapy (brachytherapy, interstitial irradiation, and intracavitary irradiation are types of internal radiotherapy.) Using internal radiotherapy, the radiation dose is concentrated in a small area, and the patient stays in the hospital for a few days. Internal radiotherapy is frequently used for cancers of the tongue, uterus, prostate, colon, and cervix.

The term “radiotherapy” or “ionizing radiation” include all forms of radiation, including but not limited to α,β, and γ radiation and ultra violet light. Radiotherapy with or without concurrent or sequential chemotherapy is an effective modality for head and neck, breast, skin, anogenital cancers, and certain nonmalignant diseases such as keloid, desmoid tumor, hemangioma, arteriovenous malformation, and histocytosis X.

In some embodiments, the methods disclosed herein reduce side effect caused by at least one other therapeutic treatment, such as radiation-induced normal tissue fibrosis or chemotherapy-induced tissue necrosis, and the methods provided herein also synergistically inhibit tumor cell growth with radiotherapy and other anti-cancer agents.

### RAD51

DNA damage causes chromosomal instability, ontogenesis, cell death, and severe dysfunction of cells. The DNA repair system is crucially important for the survival of living cells. The two major DNA repair mechanisms involved in the repair of double stranded DNA breaks are homologous recombination (HR) and non-homologous end joining (NHEJ). The eukaryotic RAD51 gene is an ortholog of Escherichia coli RecA, and the gene product RAD51 protein plays a central role in homologous recombination.

Many therapeutic treatments, such as anti-cancer agents, exert their therapeutic effects through their capability of producing DNA damage to cells. If the cells, such as cancer cells, have active DNA repair mechanisms, the therapeutic effects of such treatments may be compromised and high dosages may be needed for achieving the desired therapeutic effects.

In some embodiments, the methods disclosed herein are used to decrease cellular DNA repair activity in a human with cancer.

In some embodiments, the methods disclosed herein decrease cellular DNA repair activity in combination therapy. In some embodiments, the methods disclosed herein interfere with a DNA repairing mechanism involving RAD51 or BRCA1.

In some embodiments, the methods disclosed herein treat cancers associated with a defect in non-homologous end joining of DNA. In some embodiments, the methods disclosed herein further comprise administering a treatment capable of damaging cellular DNA.

The defect in non-homologous end joining of DNA comprises a defect in a gene selected from the group consisting of: Ku70, Ku80, Ku86, Ku, PRKDC, LIG4, XRCC4, DCLRE1C, and XLF. In one aspect, the cancer is selected from Burkitt's lymphoma, chronic myelogenous leukemia, and B-cell lymphoma. In one aspect, the cancer is described herein.

In some embodiments, the methods disclosed herein are used in the treatment of an alternative lengthening of telomere (ATL) positive cancer in a human.

Additional combination therapies, treatment strategies, and the like that include inhibiting RAD51 activity (e.g. an HDAC inhibitor (e.g. abexinostat)) are disclosed in US patent publication number 20080153877 and WO 08/082856 (both of which are herein incorporated by reference).

### Kits/Articles of Manufacture

For use in the therapeutic methods of use described herein, kits and articles of manufacture are also described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Packaging materials for use in packaging pharmaceutical products include, e.g., U.S. Pat. Nos. 5,323,907, 5,052,558 and 5,033,252. Examples of pharmaceutical packaging materials include, but are not limited to, blister packs, bottles, tubes, pumps, bags, containers, bottles, and any packaging material suitable for a selected formulation and intended mode of administration and treatment. A wide array of formulations of the compounds and compositions provided herein are contemplated.

Such kits optionally comprise an identifying description or label or instructions relating to its use in the methods described herein.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert.

### EXAMPLES

These examples are provided for illustrative purposes only and not to limit the scope of the claims provided herein.

### Synthesis of Abexinostat

Abexinostat was prepared as outlined in Example 7 of U.S. Pat. No. 7,276,612, the contents of which are incorporated herein by reference in its entirety.

### Example 1

### IV Solution of Abexinostat HCl

Abexinostat HCl was formulated as an intravenous (IV) solutions for initial clinical trials in humans. The IV solution is an aqueous solution formulation intended for infusion administration after dilution with isotonic saline. Each single use vial contains 25 mL of a 5 mg/mL (0.5%) solution of abexinostat HCl in isotonic saline and 50 mM lactate buffer, pH 4.0-4.5. All the excipients in the clinical formulations are compendial and are commonly used in parenteral formulations. The quantitative composition of the formulation is given in Table 1. The recommended storage condition is 2-8° C.

TABLE 1
Quantitative Composition of IV Solution (5 mg/mL)

| | Percent | mg/g | Typical Batch |
|---|---|---|---|
| Ingredient | (% w/w) | (w/w) | (57.5 kg) |
| Abexinostat HCl | 0.5 | 5.0 | 0.288 kg |
| Lactic acid | 0.45 | 4.5 | 0.259 kg |
| Sodium chloride | 0.665 | 6.65 | 0.382 kg |
| Water for injection | — | — | Q.S. to |
| | | | volume |
| 1N sodium hydroxide* and/or | — | — | Q.S. to pH |
| 1N hydrochloric acid* Q.S. | | | |
| to pH 4.0-4.5 ± 0.2 | | | |

### Example 2

### Immediate Release Capsules

Immediate release capsules are formulated by mixing abexinostat HCl with microcrystalline cellulose, lactose, and magnesium stearate and then adding the mixture into gelatin capsules (see Table 2). The capsules are manufactured in two strengths. A 20 mg dosage strength includes 20 mg of abexinostat HCl in a size 4 Swedish orange hard gelatin capsule. A 100 mg dosage strength includes 100 mg of abexinostat HCl in a size 2 dark green hard gelatin capsule. The capsules are packaged in 30 cc HDPE bottles and sealed with an induction seal and capped with a child resistant screw top cap. The 20 mg dosage strength is packaged at 50 capsules per bottle. The 100 mg dosage strength is packaged at 30 capsules per bottle. The bottles are stored at controlled room temperature 20-25° C. (68-77° F.).

TABLE 2
Immediate Release Capsules

| | Quality | | | |
|---|---|---|---|---|
| Component | Standard | Mg/Capsule | | Function |
| Abexinostat HCl | Manufac- | 20 mg^{(a)} | 100 mg^{(a)} | Active |
| | turer's | | | Pharmaceutical |
| | Specification | | | Ingredient |
| Avicel PH113 | NF | 68 mg | 76 mg | Disintegrant |
| (microcrystalline | | | | |
| cellulose) | | | | |
| Lactose, | NF | 15.7 mg | 17.6 mg | Diluent |
| Anhydrous | | | | |
| Magnesium | NF | 1.3 mg | 1.5 mg | Lubricant |
| Stearate | | | | |

^{(a)}The quantity of abexinostat per capsule is adjusted for water content and purity.

### Example 3

### Multiparticulate Pulsatile Formulation with Timed Release

80 grams of sodium chloride and 24 grams of polyvinylpyrrolidone are dissolved in 1.2 kilograms of water and 400 grams of pulverized abexinostat HCl are suspended therein.

In a fluidized bed coater, 400 grams of starch/sugar seeds (30/50 mesh) are suspended in warm air and spray coated with the abexinostat HCl suspension until the seeds are uniformly coated with the desired drug potency.

Magnesium stearate in isopropyl alcohol is mixed with Eudragit NE30D (Rohm Pharma of Weiterstadt, Germany), in a proportion of two to 1 of dried polymer to magnesium stearate. A sufficient amount of the polymer suspension is sprayed onto the active cores to provide a particular film coating thickness to achieve a particular lag time and rate of release for a population of pellets. The final coated pellets are dried at 50° C. for 2 hours to assure complete removal of moisture to stabilize the core contents.

The procedure is repeated with at least one more batch using a different coating thickness to have a different lag time and rate of release. In this example, two populations are prepared, one with a 10% weight gain and one with a 30% weight gain of coating. Unit doses are prepared by mixing the two populations together in predetermined proportions and filling capsules with the mixture.

After oral administration of a unit dose to a human, the first population of pellets does not begin to release abexinostat until an initial lag time of about 2-3 hours has elapsed. The second population of pellets does not begin to release abexinostat until an initial lag time of about 6-7 hours has elapsed. The mean release time (the time when half of the drug has been released) of each population of pellets should be separated from one another by at least 3-4 hours.

Fluidized bed coaters are well known in the art, however other coating apparatus and methods well known in the art may be used instead.

### Example 4

### Alternative Multiparticulate Pulsatile Formulation with Timed Release

The active cores are prepared as in Example 3. Magnesium stearate and triacetin plasticizer are mixed with Eudragit RS 30D suspension in a dry weight ratio of 1:0.6:2. The polymer suspension is coated on the cores as in Example 3, preparing a plurality of populations, each having a particular coating thickness to provide a particular lag time and rate of release of drug in an aqueous environment of use.

The different population of pellets are mixed and the mixture used to fill capsules as described in Example 3.

### Example 5

### Pulsatile Formulation—Tablets in Capsule

A pulsatile release dosage form for administration of abexinostat HCl is prepared by (1) formulating two individual compressed tablets, each having a different release profile, followed by (2) encapsulating the two tablets into a gelatin capsule and then closing and sealing the capsule. The components of the two tablets are as follows.

TABLE 3
Tablet 1 (Without Coating)

| | | Amount per |
|---|---|---|
| Component | Function | tablet |
| abexinostat HCl | Active agent | 20.0 mg |
| Dicalcium phosphate dihydrate | Diluent | 38.5 mg |
| Microcrystalline cellulose | Diluent | 38.5 mg |
| Sodium starch glycolate | Disintegrant | 2.4 mg |
| Magnesium Stearate | Lubricant | 0.6 mg |

The tablets are prepared by wet granulation of the individual drug particles and other core components as may be done using a fluid-bed granulator, or are prepared by direct compression of the admixture of components. Tablet 1 is an immediate release dosage form, releasing the active agent completely within 1-2 hours following administration.

Half of the immediate release tablets are coated with Delayed Coating No. 1 to provide Tablet 2. Tablet 2 delays the release of abexinostat HCl by about 3-5 hours after administration. Half of the immediate release tablets are coated with Delayed Coating No. 2 to provide Tablet 3. Tablet 3 delays the release of abexinostat HCl by about 4-9 hours after administration. The coating is carried out using conventional coating techniques such as spray-coating or the like.

TABLE 4
Tablet 2 (with Coating)

| Component | Function | Weight |
|---|---|---|
| Tablet 1 | “Core” containing the active agent | 100.0 mg |
| Eudragit RS30D | Delayed release coating material | 8.0 mg |
| Talc | Coating component | 6.0 mg |
| Triethyl citrate | Coating component | 2.0 mg |

TABLE 5
Tablet 3 (with Coating)

| Component | Function | Weight |
|---|---|---|
| Tablet 1 | “Core” containing the active agent | 100.0 mg |
| Eudragit RS30D | Delayed release coating material | 12 mg |
| Talc | Coating component | 7 mg |
| Triethyl citrate | Coating component | 3.0 mg |

Oral administration of the capsule to a patient should result in a release profile having two pulses, with initial release of abexinostat HCl occurring about 3-5 hours following administration, and release of abexinostat from the second tablet occurring about 7-9 hours following administration.

### Example 6

### Pulsatile Formulation—Beads in Capsule or Tablet

The method of Example 5 is repeated, except that drug-containing beads are used in place of tablets. Immediate release beads are prepared by coating an inert support material such as lactose with the drug. The immediate release beads are coated with an amount of enteric coating material sufficient to provide a drug release-free period of about 3-5 hours. A second fraction of beads is prepared by coating immediate release beads with a greater amount of enteric coating material, sufficient to provide a drug release-free period of about 7-9 hours. The two groups of coated beads are encapsulated as in Example 5, or compressed, in the presence of a cushioning agent, into a single pulsatile release tablet.

### Example 7

### Sustained Release Tablet

Sustained release tablets of abexinostat are prepared by first preparing a sustained release excipient. The sustained release excipient is prepared by dry blending the requisite amounts of xanthan gum, locust bean gum, a pharmaceutically acceptable hydrophobic polymer and an inert diluent in a high-speed mixer/granulator for 2 minutes. While running choppers/impellers, the water was added and the mixture was granulated for another 2 minutes. The granulation was then dried in a fluid bed dryer to a loss on drying weight (“LOD”) of between 4 and 7%. The granulation was then milled using 20 mesh screens. The ingredients of the sustained release excipients are set forth in Table 6 below:

TABLE 6
Sustained Release Excipient Mixture

| Component | % by Weight |
|---|---|
| Xanthan Gum | 10 |
| Locust Bean Gum | 10 |
| Carboxymethylcellulose | 30 |
| Dextrose | 50 |
| Water | 23* |

*removed during processing

Next, the sustained release excipient prepared as detailed above is dry blended with a desired amount of abexinostat in a V-blender for 10 minutes. A suitable amount of tableting lubricant Pruv® (sodium stearyl fumarate, NF) for the following examples is added and the mixture is blended for another 5 minutes. This final mixture is compressed into tablets, each tablet containing 10% by weight, of abexinostat. The tablets produced weighed 500 mg (Diameter is ⅜ inches; hardness is 2.6 Kp). The proportions of the tablets are set forth in Table 7 below.

TABLE 7
Sustained Release Tablets

| Component | % by Weight |
|---|---|
| sustained release excipient mixture of Table 6 | 88.5 |
| abexinostat | 10 |
| Sodium Stearyl Fumarate | 1.5 |

Dissolution tests are then carried out on the tablets. The dissolution tests are conducted in an automated USP dissolution apparatus (Paddle Type II, pH 7.5 buffer, 50 rpm in 500 mL). The tablets should release about 30% of abexinostat by 2 hours, followed by a sustained release such that about 98% of abexinostat is released at the end of 12 hours.

### Example 8

### Coated Sustained Release Tablet

A sustained release excipient was prepared as described above by dry blending the requisite amounts of xanthan gum, locust bean gum and an inert diluent. An extra 2 minutes of granulation was used after the addition of the components (for 4 total minutes of post-addition granulation). Ethylcellulose aqueous dispersion was substituted for water in the above methods. The components of the sustained release excipient is described in Table 8.

TABLE 8
Sustained Release Excipient

| Component | % by Weight |
|---|---|
| Xanthan Gum | 12 |
| Locust Bean Gum | 18 |
| Dextrose | 65 |
| Ethylcellulose Aqueous Dispersion | 5* |

*Ethylcellulose Aqueous Dispersion contains approx. 25% by weight of solids. The amount added to the formulation (i.e. 5%) is solids only.

The xanthan gum and locust bean gum are dry blended in a V-blender for 10 minutes, the dextrose is added and the mixture blended for another 5 minutes. The ethylcellulose aqueous dispersion is then added, followed by an additional 5 minutes of blending. The resulting granulation is then compressed into tablets with sodium stearyl fumarate, as a tableting lubricant. The tablets are then coated with additional ethylcellulose aqueous dispersion. To accomplish this, ethylcellulose (Surelease®, 400 g) is mixed with water (100 g) to form an aqueous suspension. Thereafter, the tablets are coated in a Keith Machinery coating pan (diameter 350 mm; pan speed 20 rpm; spray-gun nozzle 0.8 mm; tablets bed temperature 40°-50° C.; charge per batch 1 kg; dry air—Conair Prostyle 1250, 60°-70° C.). The tablets are coated to a weight gain of about 5%. The tablets should weigh about 500 mg. The proportions of the tablets are set forth in Table 9 below:

TABLE 9
Coated Sustained Release Tablets

| Component | % by Weight |
|---|---|
| sustained release excipient mixture of Table 8 | 83.5 |
| abexinostat | 10 |
| Ethylcellulose | 5 |
| Sodium Stearyl Fumarate | 1.5 |

The dissolution tests are conducted in an automated USP dissolution apparatus in such a way as to model passage through the gastrointestinal tract. The coated tablets should not release more than 10% abexinostat during the first 1-2 hours, and then should release abexinostat at a steady rate such that about 90% to 100% of abexinostat is released after 12 hours.

### Example 9

### In Vitro Release Profiles

The dissolution profiles are obtained using the United States Pharmacopeia Apparatus I at 37° C. and 100 RPM. The dissolution media is varied with time beginning with 0.1N HCl for 0-2 hours. From 2 to 4 hours the media is pH 6.5 phosphate buffer and from 4 to 24 hours the media was PH 7.5 phosphate buffer.

Alternatively, dissolution profiles are performed using a USP Type III (VanKel Bio-Dis II) apparatus.

### Example 10

### In vitro Fed/Fast Dissolution Protocol

The test formulations are evaluated under a variety of dissolution conditions to determine the effects of pH, media, agitation and apparatus. Dissolution tests are performed using a USP Type III (VanKel Bio-Dis II) apparatus. In order to determine the differences, if any, in dissolution kinetics between a fed state and a fasting state for the series of formulations, in vitro dissolution experiments are carried out in a solution containing 30% peanut oil (“fed”) to model a gastrointestinal tract with a typical dietary fat load. The control determined the dissolution rates in a solution lacking the fat load (“fasted”). The pH-time protocol (ranging from acid to alkaline to model digestive processes) is set forth below in Table 10, below. Agitation is 15 cpm. Volume of the sample tested is 250 mL.

TABLE 10
Fed/Fast Dissolution Protocol

Apparatus Media

| “Fed” | “Fasted” | Time | pH |
|---|---|---|---|
| 30% Peanut Oil | No Peanut oil | 0-1 hour | 1.5 |
| 30% Peanut Oil | No Peanut oil | 1-2 hour | 3.5 |
| 30% Peanut Oil | No Peanut oil | 2-4 hour | 5.5 |
| 30% Peanut Oil | No Peanut oil | 4-12 hour | 7.5 |

An enteric coating on the tablet is expected to provide a tablet that provides dissolution rates that are not significantly different in the fasted and fed states.

### Example 11

### Phase 1 Trial

### Study Objectives

Determine the safety, tolerability and maximum tolerated dose (MTD) of pazopanib HCl in combination with abexinostat HCl in patients with advanced solid tumors

Characterize the pharmacokinetics of abexinostat HCl, pazopanib HCl, and the combination of the two.

Evaluate preliminary efficacy using clinical benefit rate=CR+PR+SD, objective response proportion, and progression-free survival.

Explore the relationship of changes in expression levels of histone acetylation in blood and biopsied tumors and expression of biomarkers including VEGF, VEGFR, HIF, and RAD51 in plasma in responders and nonresponders.

Explore variations of single-nucleotide polymorphisms (SNPs) in relationship to potential toxicities.

Evaluate functional imaging using FLT PET (3′deoxy-3′-18F-Fluorothymidine positron emission tomography) to measure changes in rates of cell division and correlation with tumor response.

### Overview of Study Design

Open label, non-randomized, dose escalation and expansion Phase I trial to evaluate the safety of the combination of abexinostat and Pazopanib and to determine the recommended Phase II dose of the combination.

Pazopanib HCl will be given once daily days 1-28 and should be taken orally without food at least one hour before or two hours after a meal. Abexinostat HCl will be given orally twice a day during dl-5, 8-12, 15-19. Each cycle will be 28 days in duration. A cycle duration is 28 days. Patients will continue on treatment until disease progression.

### Inclusion Criteria

Phase Ia: Patients must have histologically or cytologically documented metastatic solid tumor malignancies.

Phase Ib: Patients must have histologically or cytologically confirmed locally advanced, unresectable or metastatic sarcoma or renal cell carcinoma.

Measurable disease by RECIST 1.1

Patients may have de novo metastatic disease, or progressed despite any number of prior therapies

Eastern Cooperative Oncology Group (ECOG) performance status of 0-1

Resolution of all chemotherapy or radiation-related toxicities to Grade 1 severity or lower except for alopecia

Patient must be at least 2 weeks or five half-lives (whichever is longer) from last standard or experimental therapy, including radiotherapy

Patients who have received prior pazopanib HCl are eligible but must not have received it in the last two weeks

### Exclusion Criteria

Patients with other untreated, current primary malignancies, other than carcinoma in situ of the cervix or non-melanoma skin cancer

History or clinical evidence of central nervous system (CNS) metastases or leptomeningeal carcinomatosis, except for individuals who have previously-treated CNS metastases, are asymptomatic, and have had no requirement for steroids or anti-seizure medication for 4 weeks prior to first dose of study drug.

Clinically significant gastrointestinal abnormalities that may increase the risk for gastrointestinal bleeding.

Corrected QT interval (QTc)>480 msecs using Friedrichs's formula

Use of medications that are known to prolong cause QT prolongation

History of any one or more of the following cardiovascular conditions within the past 6 months:
a. Cardiac angioplasty or stenting
b. Myocardial infarction
c. Unstable angina
d. Coronary artery bypass graft surgery
e. Symptomatic peripheral vascular disease

Poorly controlled hypertension [defined as systolic blood pressure (SBP) of ≧140 mmHg or diastolic blood pressure (DBP) of ≧90 mmHg].

History of cerebrovascular accident including transient ischemic attack (TIA), pulmonary embolism or untreated deep venous thrombosis (DVT) within the past 6 months.
a. Note: Patients with recent DVT who have been treated with therapeutic anticoagulation for at least 6 weeks are eligible

Any serious and/or unstable pre-existing medical, psychiatric, or other condition that could interfere with subject's safety, provision of informed consent, or compliance to study procedures

Unable or unwilling to discontinue use of prohibited medications for at least 14 days or five half-lives of a drug (whichever is longer) prior to the first dose of study drug and for the duration of the study

### Patient Cohorts and Dose Escalation Rules

This trial proposes to use abexinostat HCl to increase efficacy and potentially reverse mechanisms of resistance to angiogenesis inhibitors, in this study, pazopanib HCl. To accommodate optimal dosing and to reach steady level of abexinostat HCl, abexinostat HCl will be taken orally twice daily on Days 1-5, 8-12, 15-19 of 28 Days. Pazopanib will be taken daily on Days 1-28 of 28 Days. Cycles will be repeated every 28 Days.

Patients will receive alternating escalating doses of abexinostat HCl and pazopanib HCl. Dose escalations will occur based on the table below. The following dose cohorts are planned, however if >2 DLT are observed in any cohort and no DLT was seen in the previous cohort, an intermediate dose level will be explored (e.g: 2 DLTs are observed at 45 mg, and no DLT at 30 mg, we will explore 35 mg).

If a DLT likely related to pazopanib HCl is observed in the first cohort, pazopanib HCl dose will be lowered first. If there is evidence that the toxicity is likely due to abexinostat HCl, the abexinostat HCl dose will be lowered to 30 mg (cohort −1).

During Phase Ia, patients must receive 20 days of pazopanib HCl (>75%) and 10 days of abexinostat HCl (>75%) during the first cycle in order to be evaluable for DLT. If therapy is delayed >14 days during the first cycle attributable to study drug, this is considered a DLT and the patient will not be replaced. If therapy is delayed due to another reason, the patient will be replaced.

| | Number | | |
|---|---|---|---|
| Cohorts | of pts | Pazopanib HCl | Abexinostat HCl |
| −1 | (6) | 400 mg po qd | 30 mg/m2 PO BID |
| | | | d 1-5, 8-12, 15-19 |
| 1* | 1 (+2) | 400 mg po qd | 45 mg/m2 PO BID |
| | | | d 1-5, 8-12, 15-19 |
| 2 | 1 (+2) | 600 mg po qd | 45 mg/m2 PO BID |
| | | | d 1-5, 8-12, 15-19 |
| 3 | 3 | 600 mg po qd | 60 mg/m2 PO BID |
| | | | d 1-5, 8-12, 15-19 |
| 4 | 3 | 800 mg po qd | 60 mg/m2 PO BID |
| | | | d 1-5, 8-12, 15-19 |
| 5 | 6 | 800 mg po qd | 75 mg/m2 PO BID |
| | | | d 1-5, 8-12, 15-19 |
| MTD | 20 each in | xxx mg po qd | xxx mg/m2 PO BID |
| | sarcoma, | | d 1-5, 8-12, 15-19 |
| | RCC, and | | |
| | other | | |

*starting dose

Starting at dose level 1, if 1 patient experiences DLT (as defined in section 4.5) that dose level will be expanded to include 2 additional patients. If the additional patients have no DLTs, the dose will be expanded to the next level. If 2/3 patients have a DLT the dose will be de-escalated to dose −1. At dose level 3, expansion part I will occur in a standard 3+3 design. Three patients will be treated at dose level 3 and 4. If 0/3 patients experience DLT, 3 patients will be treated at the next dose level. If DLT attributable to the treatment is experienced in 1/3 patients, three more patients (for a total of six patients) will be treated at that dose level. If no additional DLT are observed at the expanded dose level (i.e. 1/6 with DLT), the dose will be escalated. Escalation will terminate as soon as two or more patients experience any DLT attributable to study drugs, at a given dose level. If dose level 5 is reached 6 patients will be enrolled. Once the MTD is defined, dose expansion part II will occur.

No intra cohort dose escalations will be permitted. Dose escalation will be followed according to the outlined escalation steps: abexinostat HCl should be started in the morning of Day 1 and continued on Days 2-5 of a 28 Day Cycle. Pazopanib will be given on Day 2 after the morning dose of abexinostat HCl in Cycle 1 only and then daily for 28 days. A four-week treatment is defined as one Cycle. Responses will be assessed after two Cycles. A medication diary by the patient will be assessed after each Cycle.

If at any dose, DLTs are observed and no DLTs were observed at the previous dose level, we may explore a dose that is intermediate after discussion with the CHR, PI and the sponsor.

There will be no more than 2 patients dosed for the first time within the same week and patients in the next higher cohorts will not be enrolled until the last patient of the lower cohort has completed the DLT period

### Estimated Patient Numbers

The total number of patients to be enrolled on the study will be between 46 and 90.

### Duration of Intervention and Evaluation

Patients will be on the study until progression of disease as defined by RECIST 1.1, intolerable toxicity, request to withdraw, or withdrawal per the Principal Investigator.

Patients will continue to be followed periodically (approximately every 6 months) through medical records, and subsequent cancer treatments, progression of cancer, and survival outcome will be updated. Follow-up will occur until death or for at least ten years.

### Dose Limiting Toxicities

This is a combination trial which may have different toxicities resulting from the pazopanib HCl and abexinostat HCl dose escalation or those resulting from the combination. Special consideration should be given to toxicities arising from the dose escalations. The rationale of this trial is to increase the efficacy of each drug by combination therapy and to reverse resistance mechanisms to angiogenesis inhibitors. Every effort should be made not to delay drug dosing. Prior approval by the Principal Investigator is required to delay dosing. If toxicities can be clearly linked to one drug only, only the offending agent should be dose-modified.

Adverse Events and other symptoms will be graded according to the NCI Common Terminology Criteria for Adverse Events Version 4.03 (NCI, CTC web site http://ctep.info.nih.gov).

A dose limiting toxicity (DLT) will be defined as any one of the following adverse events 31 occurring during Cycle 1 when association to therapy that is part of this study is related or possibly related:

Hematologic Dose-Limiting Toxicity
a. Grade 4 neutropenia lasting for ≧7 days in duration despite growth factor support. GCSF (Filgrastim) or Pegylated-GCSF (Neulasta) may be administered after day 7 Cycle 1 to treat an ANC≦1000, and prophylactically after Cycle 1 at the discretion of the treating physician. When administered, this does not constitute a DLT.
b. Grade 4 neutropenia with fever >38.5° C. and infection requiring antibiotic or anti-fungal treatment
c. Grade 4 thrombocytopenia (≦25.0×109/L)
d. Grade 3 thrombocytopenia complicated by bleeding and/or requiring platelet or blood transfusion

Non-hematologic dose-limiting toxicity—this will be defined as any Grade >3 non-hematologic toxicity, with specific exceptions.

The following will also be considered DLT:
a. Symptomatic bradycardia
b. Persistent increases in QTc interval (>60 milliseconds from baseline and/or >500 ms)
c. Treatment delay of greater than 14 days
d. Failure to administer ≧75% of the planned study drugs during cycle 1 as a result ≧Grade 2 treatment-related toxicity
e. Subjects who fail to complete the first cycle due to reasons other than toxicity will be classified as not evaluable for toxicity, and will be replaced. No dose reductions can occur within the DLT window.

### Maximum Tolerated Dose

The maximum tolerated dose (MTD) will be defined as the highest tested dose level at which less than 33% of patients experience DLT in Cycle 1.

Visit Schedule and Assessments

| | Pre- | Cycle 1 Day 1-28 | | | |
|---|---|---|---|---|---|
| Test/Study | study | Week 1 | Week 2 | Week 3 | Week 4 |
| History and | X¹ | X¹ | | | |
| drug diary | | | | | |
| Physical | X¹ | X¹ | | | |
| Exam³ | | | | | |
| Toxicity | X¹ | X | X | X | X |
| assessment⁴ | | | | | |
| CBC, Diff.⁵ | X¹ | X¹ | | | |
| Chemistry⁶ | X¹ | X¹ | X^{Error! Bookmark not defined.} | | |
| TSH and | X | | | | |
| T4⁷ | | | | | |
| INR/PT⁸ | X¹ | | | | |
| Urine | X¹ | | | | |
| protein⁹ | | | | | |
| Echo/ | X | | | | |
| MUGA¹⁰ | | | | | |
| EKG^{11, 12} | X | X^{Error! Bookmark not defined.} | X^{Error! Bookmark not defined.} | X^{Error! Bookmark not defined.} | X^{Error! Bookmark not defined.} |
| Pregnancy | X | | | | |
| Test¹³ | | | | | |
| Staging | X | | | | |
| Studies¹⁴ | | | | | |
| Pazopanib | | X | | | X |
| PK¹⁵ | | | | | |
| Abexinostat | | X | | | |
| PK¹⁶ | | | | | |
| PD | X¹⁸ | X^{18, 19} | X¹⁸ | | |
| markers¹⁷ | | | | | |
| FLT/PET²¹ | | X | | | |
| Tumor | X | X | | | |
| biopsy²² | | | | | |
| Test/ | Cycle 2 Day 1-28 | | | | Every 8 wks until 6 |
| Study | Week 1 | Week 2 | Week 3 | Week 4 | months on study |
| History and | X^{Error! Bookmark not defined.} | | | | |
| drug diary | | | | | |
| Physical | X^{Error! Bookmark not defined.} | | | | |
| Exam³ | | | | | |
| Toxicity | X | | | | |
| assessment⁴ | | | | | |
| CBC, Diff.⁵ | X | | | | |
| Chemistry⁶ | X | | | | |
| TSH and | X | | | | X |
| T4⁷ | | | | | |
| INR/PT⁸ | | | | | |
| Urine | | | | | |
| protein⁹ | | | | | |
| Echo/ | | | | X | |
| MUGA¹⁰ | | | | | |
| EKG^{11, 12} | X^{Error! Bookmark not defined.} | | | | |
| Pregnancy | X | | | | |
| Test¹³ | | | | | |
| Staging | | | | X | X |
| Studies¹⁴ | | | | | |
| Pazopanib | | | | | |
| PK¹⁵ | | | | | |
| Abexinostat | | | | | |
| PK¹⁶ | | | | | |
| PD | X^{Error! Bookmark not defined.} | | | | |
| markers¹⁷ | | | | | |
| FLT/ | | | | X | |
| PET²¹ | | | | | |
| Tumor | | | | | |
| biopsy²² | | | | | |
| | | Test/ | Every 8 wks after 6 | Prior to Day | |
| | | Study | months on study | 1 of each Cycle | End of Treatment |
| | | History and | | X^{Error! Bookmark not defined.} | X |
| | | drug diary | | | |
| | | Physical | | X^{Error! Bookmark not defined.} | X |
| | | Exam³ | | | |
| | | Toxicity | | X | X |
| | | assessment⁴ | | | |
| | | CBC, Diff.⁵ | | X | X |
| | | Chemistry⁶ | | X | X |
| | | TSH and | X | | X |
| | | T4⁷ | | | |
| | | INR/PT⁸ | | | |
| | | Urine | | X | |
| | | protein⁹ | | | |
| | | Echo/ | ? | | X |
| | | MUGA¹⁰ | | | |
| | | EKG^{11, 12} | | X^{Error! Bookmark not defined.} | X |
| | | Pregnancy | | X | X |
| | | Test¹³ | | | |
| | | Staging | X | | |
| | | Studies¹⁴ | | | |
| | | Pazopanib | | | |
| | | PK¹⁵ | | | |
| | | Abexinostat | | | |
| | | PK¹⁶ | | | |
| | | PD | | X^{Error! Bookmark not defined.} | X |
| | | markers¹⁷ | | | |
| | | FLT/ | | | |
| | | PET²¹ | | | |
| | | Tumor | | | |
| | | biopsy²² | | | |

¹Pre-study tests, history and physical exam may be used for Day 1 tests if within 2 weeks and no significant changes have occured
²D1 physical exam and history in subsequent Cycles may be done within 7 Days prior to next Cycle
³Physical Exam includes ECOG status and vital signs
⁴Toxicity will be assessed by CTCAE v4.03
⁵Hemoglobin, hematocrit, platelets, total white blood cell count (WBC) and differential
⁶BUN, creatinine, sodium, potassium, chloride, CO2 (HCO3), glucose, calcium, albumin, total protein, total bilirubin, alkaline phosphatase, LDH (melanoma only), AST/SGOT, ALT/SGPT, phosphorous, magnesium If total bilirubin is greater than the upper limit of normal, direct and indirect bilirubin should be performed. Biochemistry tests should be obtained after patient has fasted, if possible. LFTs including total bilirubin, alkaline phosphatase, LDH (melanoma only), AST/SGOT, ALT/SGPT should also be obtained during Cycle 1 Week 2.
⁷Thyroid functions tests: TSH, FT4 every 8 weeks
⁸For patients taking warfarin, the coagulation profile includes a prothrombin time or International Normalized Ratio (INR)
⁹Urine protein should be measured by protein quantification in urinalysis
¹⁰MUGA or ECHO should be performed at baseline and the end of Cycle 2 (±1 week) and only repeated with subsequent cycles if EF changes ±10%.
¹¹Cycle 1 weekly triplicate EKGs at two timepoints: pre-abexinostat HCl and 3 hours (±15 min.) post- abexinostat HCl.
¹²Cycles ≧ 2: Single EKGs if no cardiac problems identified on pre-dose Day 1 EKG.
¹³For women of childbearing potential. Pregnancy test will be repeated after each two Cycles if clinically indicated.
¹⁴Baseline evaluations should be performed not more than 30 Days prior to the beginning of the treatment.
¹⁵Pazopanib PK: final schedule TBD Phase Ia only
Day 3: predose, after dose: 30 minutes, 2, 4, 8, 24 hours
Day 8: predoses (with abexinostat HCl), after doses: 30 minutes, 1 hour, 2 hours, 4 hours, 8 hours
Day 22: predose, after dose: 30 minutes, 2, 4, 8, 24 hours
¹⁶abexinostat HCl PK: final schedule TBD Phase Ia only
Day 1: predose, after dose: 30 minutes, 1, 2, 4, 6, 8 24 hours
Day 8: see #15
¹⁷PD markers will include histone acetylation, expression of VEGF, VEGFR, HIF, RAD51, pharmacogenomics
¹⁸PD markers for abexinostat HCl:
Pre-treatment: up to 10 Days prior
Day 1: 2-hours (+15 min) post abexinostat HCl
Day 8: pre- and 2-hours (+15 min) post abexinostat HCl
¹⁹PD markers for pazopanib HCl: plasma to be drawn with each Cycle
²⁰Pharmacogenomics: whole blood to be drawn Cycle 1 Day 1
²¹Tumor FNA:
Day 1 (up to 10 Days prior), and Day 5 at 120 min (+30 min) post abexinostat HCl.
*Tumor FNA or tumor biopsies are optional for dose escalation, mandatory for dose expansion*
²²FLT PET (3′deoxy-3′-18F-Fluorothymidine positron emission tomography) can be performed with baseline imaging and then prior to Cycle 2 with follow-up imaging.

| | | Cycle 1 (Days 1-28) | | | | |
|---|---|---|---|---|---|---|
| PK schedule | Dosing | D1 | D2 | D3 | D22 | D23 |
| Pazopanib | Once a | | | X | X | X |
| (po) | Day | | | | | |
| PCI-24781 | Twice a | X | X | X | | |
| (po) | Day | | | | | |

Pazopanib schedule: pre, 30 min, hr 2, 4, 8, 24
Abexinostat HCl schedule: pre, 30 min, hr 2, 4, 8, 24

### Efficacy Assessments

Criteria for response, progression and relapse

Response and progression will be evaluated in this study using the new international criteria proposed by the Response Evaluation Criteria in Solid Tumors (RECIST) Committee 33. Changes in only the largest diameter (unidimensional measurement) of the tumor lesions are used in RECIST 1.1. Note: Lesions are either measurable or non-measurable using the criteria provided below. The term “evaluable” in reference to measurability will not be used because it does not provide additional meaning or accuracy.

For the purposes of this study, patients should be evaluated for response every 8 weeks, prior to the start of odd-numbered Cycles after Cycle 1. In addition to a baseline scan, confirmatory scans should also be obtained >4 weeks following initial documentation of objective response.

### Evaluation of Target Lesions

Complete Response (CR): Disappearance of all target lesions

Partial Response (PR): At least a 30% decrease in the sum of the longest diameter (LD) of target lesions, taking as reference the baseline sum LD

Progressive Disease (PD): At least a 20% increase in the sum of the LD of target lesions, taking as reference the smallest sum LD recorded since the treatment started or the appearance of one or more new lesions

Stable Disease (SD): Neither sufficient shrinkage to qualify for PR nor sufficient increase to qualify for PD, taking as reference the smallest sum LD since the treatment started

### Tumor Samples and PBMCs

Tumor samples by fine needle aspirations (FNA) will be obtained by the study cytopathologist based on the schedule of assessments post-abexinostat HCl. Diff-Quick air-dry method (FNA) at time of aspiration will be used by the study cytopathologist to confirm the presence of tumor cells in the specimen. An accessible lesion for the purpose of this study is defined as a subcutaneous nodule or lymph node or a lesion accessible to FNA with CT guidance with low risk to the patient (Includes CT/ultrasound guided FNA of lymph nodes in the neck, axilla, groin, tumor masses in the breast, liver or adrenals). This decision will be at the discretion of the treating physician in consultation with the principal investigator. If no tumor nodule is visible and/or palpable or accessible as defined above, then no biopsy will be done.

Tissues will be evaluated for the effects of PCI24781 on tumor and PBMC histone acetylation. PBMCs and tumor aspirates will be processed in Pamela Munster's laboratory at UCSF using immunofluorescence and Western Blot analysis (IF) analysis. Cells will also be stained for HDAC enzyme expression.

Other correlative study methods will be added later.

### Safety Assessments

Safety assessments will consist of monitoring and recording all adverse events and serious adverse events, the regular monitoring of hematology, blood chemistry and urine values, vital signs, ECOG performance status, and the regular physical examinations and ECG assessments.

Adverse events will be assessed according to the Common Toxicity Criteria for Adverse Events (CTCAE) version4.03.

A serious adverse event is any adverse drug experience occurring at any dose that:
a. results in death;
b. is life-threatening;
c. results in in-patient hospitalization or prolongation of existing hospitalization (admissions for elective surgeries or procedure do not qualify);
d. results in a persistent or significant disability/incapacity; or
e. results in congenital anomaly/birth defect.

An adverse event is the appearance or worsening of any undesirable sign, symptom, or medical condition occurring after starting the study drug even if the event is not considered to be related to study drug. Medical conditions/diseases present before starting study drug are only considered adverse events if they worsen after starting study drug. Abnormal laboratory values or test results constitute adverse events only if they induce clinical signs or symptoms, are considered clinically significant, or require therapy.

The occurrence of adverse events should be sought by non-directive questioning of the patient at each visit during the study. Adverse events also may be detected when they are volunteered by the patient during or between visits or through physical examination, laboratory test, or other assessments. As far as possible, each adverse event should be evaluated to determine: the severity grade (mild, moderate, severe) or (grade 1-4); its relationship to the study drug(s) (suspected/not suspected); its duration (start and end dates or if continuing at final exam); action taken (no action taken, study drug dosage adjusted/temporarily interrupted, study drug permanently discontinued due to this adverse event, concomitant medication taken, non-drug therapy given, hospitalization/prolonged hospitalization); and whether it constitutes a serious adverse event (SAE).

All adverse events should be treated appropriately. Such treatment may include changes in study drug treatment including possible interruption or discontinuation, starting or stopping concomitant treatments, changes in the frequency or nature of assessments, hospitalization, or any other medically required intervention. Once an adverse event is detected, it should be followed until its resolution, and assessment should be made at each visit (or more frequently, if necessary) of any changes in severity, the suspected relationship to the study drug, the interventions required to treat it, and the outcome.

Information about all serious adverse events will be collected and recorded.

### Endpoints

DLT will be assessed by monitoring for adverse events, scheduled laboratory assessments, vital sign measurements, ECGs, and physical examinations. The severity of the toxicities will be graded according to the NCI CTCAE v4.03, published 14 Jun. 2010. Adverse events and clinically significant laboratory abnormalities (meeting Grade 3, 4, or 5 criteria according to CTCAE) will be summarized by maximum intensity and relationship to study drug for each treatment group. Safety will be assessed weekly for the first 4 weeks and then every 4 weeks. Simple descriptive statistics will be utilized to display the data on toxicity seen from the combination of pazopanib HCl and abexinostat HCl.

Noncompartmental pharmacokinetics of abexinostat HCl, pazopanib HCl, and the combination will be assessed by measuring and calculating the volume of distribution (Vd), bioavailability (F), clearance (CL), half-life (t1/2), and area under the curve (AUC).

Clinical Benefit Rate=CR+PR+SD. Evaluated by imaging criteria RECIST 1.1

Objective response rate. Will be calculated as a proportion, the number of patients by best response (who had clinical benefit) divided by the total number of patients on study.

Progression-free survival. Time to progression will be calculated as the time from study enrollment until the time of disease relapse, progression, or death from any cause, or until last contact if no relapse, progression or death occurred.

Overall survival. OS time will be calculated as the time from study enrollment until the time of death from any cause, or until last contact if the patient did not die.

Histone acetylation as measured by changes in HDAC1, HDAC2, HDAC3, and HDAC6 expression in PBMC and tumor biopsies

Other PD biomarkers: plasma for VEGF, VEGFR, HIF, and RAD51 expression

Pharmacogenomics: one time collection of blood for evaluation of SNP variations and correlation with toxicities

Changes in FLT PET (3′deoxy-3′-18F-Fluorothymidine positron emission tomography)

### Example 11

### In Vitro Assay of Effects of Pazopabnib+Abexinostat

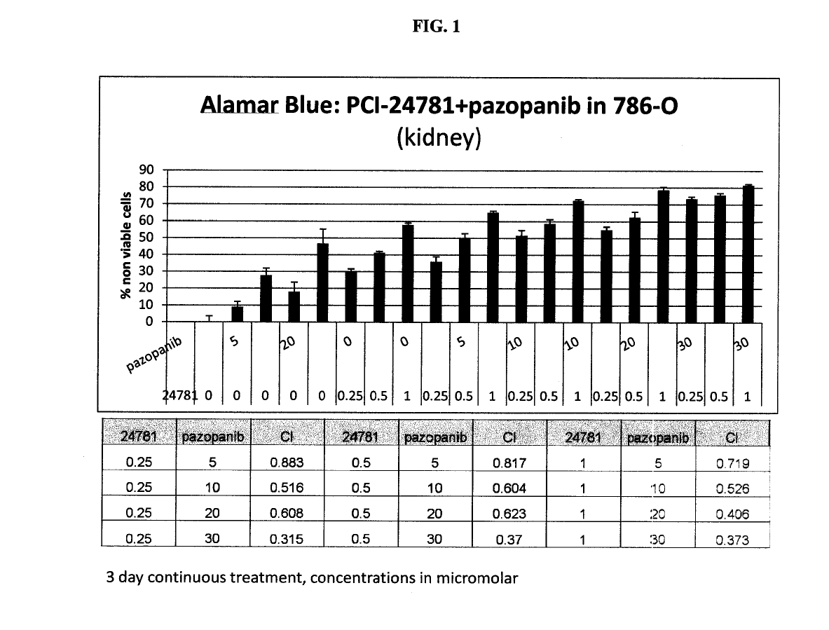

The effects of the combination of pazopanib+abexinostat (PCI-24781) were assayed in 786-O human kidney carcinoma cells. Results are presented in FIG. 1. The combination was administered to cells for three continuous days, after which alamarBlue levels were measured.

### Example 12

### In Vitro Assay of Effects of Pazopabnib+Abexinostat

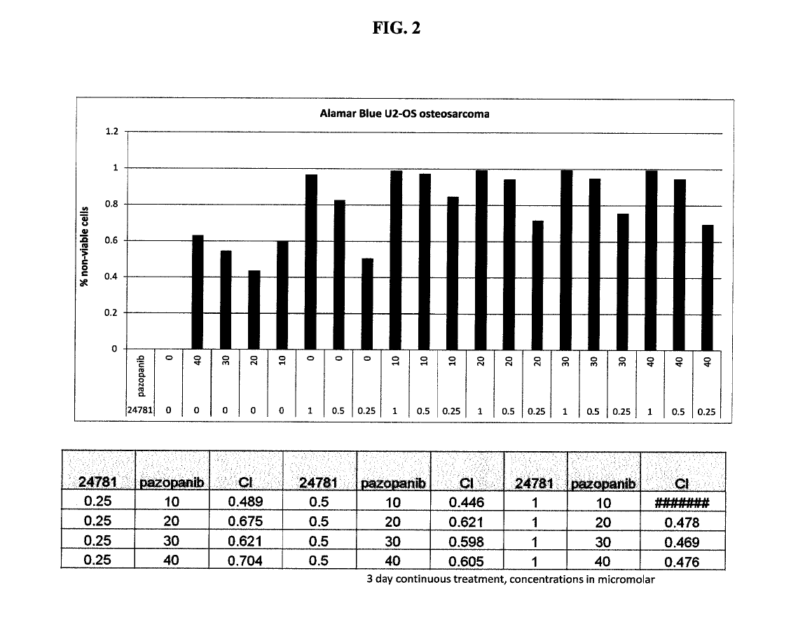

The effects of the combination of pazopanib+abexinostat (PCI-24781) was assayed in U2-OS osteosarcoma cells. Results are presented in FIG. 2. The combination was administered to cells for three continuous days, after which alamarBlue levels were measured.

The examples and embodiments described herein are for illustrative purposes only and various modifications or changes suggested to persons skilled in the art are to be included within the spirit and purview of disclosure and scope of the appended claims. As will be appreciated by those skilled in the art, the specific components listed in the above examples may be replaced with other functionally equivalent components, e.g., diluents, binders, lubricants, fillers, coatings, and the like.

1.-18. (canceled)

19. A method of treating a cancer in an individual in need thereof, comprising co-administering to the individual (a) a cycle of abexinostat or a salt thereof, and (b) an antiangiogenic agent.

20. The method of claim 19, wherein the antiangiogenic agent is pazopanib or a salt thereof.

21. The method of claim 20, wherein the method reduces resistance to the antiangiogenic agent; delays the development of resistance to the antiangiogenic agent; delays the onset of the cancer becoming refractory to the antiangiogenic agent; prolongs the usefulness of the antiangiogenic agent; allows use of the antiangiogenic agent in the treatment of cancers that generally develop, or have developed, resistance to the antiangiogenic agent; increases patient response to the antiangiogenic agent; increases cellular response to the antiangiogenic agent; decreases the effective dosage of the antiangiogenic agent; or any combination thereof.

22. The method of claim 20, wherein the salt of abexinostat is abexinostat HCl.

23. The method of claim 20, wherein abexinostat, or a salt thereof, and the antiangiogenic agent are administered separately, concurrently or sequentially.

24. The method of claim 20, wherein the subject is in an interdigestive state.

25. The method of claim 20, wherein the abexinostat, or a salt thereof, and the antiangiogenic agent, are administered one hour before a meal or 2 hours after a meal.

26. The method of claim 20, wherein the cycle of abexinostat, or a salt thereof, is 5 days.

27. The method of claim 20, wherein at least one dose of abexinostat, or a salt thereof, is administered each day of the abexinostat cycle.

28. The method of claim 27, wherein the dose of abexinostat, or a salt thereof, is sufficient to maintain an effective plasma concentration of abexinostat, or the salt thereof, in the individual for at least about 6 consecutive hours to about 8 consecutive hours.

29. The method of claim 20, comprising administering a first dose of abexinostat, or a salt thereof, and a second dose of abexinostat, or a salt thereof, 4 to 8 hours apart.

30. The method of claim 20, wherein the cancer is a hematological cancer, solid tumor or a sarcoma.

31. The method of claim 20, wherein the cancer is a solid tumor.

32. The method of claim 31, wherein the cancer solid tumor is a metastatic solid tumor or an advanced solid tumor.

33. The method of claim 20, wherein the cancer is a sarcoma.

34. The method of claim 20, wherein the cancer is soft tissue sarcoma.

35. The method of claim 20, wherein the cancer is renal cell carcinoma or ovarian cancer.

36. The method of claim 20, wherein the cancer is resistant to the antiangiogenic agent; partially resistant to the antiangiogenic agent; or refractory to the antiangiogenic agent.

37. The method of claim 20, further comprising administering at least one additional therapy selected from anti-cancer agents, anti-emetic agents, radiation therapy, or combinations thereof.
